(19) United States
(12) Patent Application Publication
(10) Pub. No.: US 2025/0236843 A1
(43) Pub. Date: Jul. 24, 2025

(54) ENGINEERED CELLS AND USES THEREOF

(71) Applicant: Ohio State Innovation Foundation, Columbus, OH (US)

(72) Inventors: Benjamin SEGAL, Columbus, OH (US); Andrew SAS, Columbus, OH (US); Andrew JEROME, Columbus, OH (US); Tom LIU, Dublin, OH (US)

(21) Appl. No.: 18/714,308

(22) PCT Filed: Nov. 30, 2022
(86) PCT No.: PCT/US2022/080673

§ 371 Date: May 29, 2024

Related U.S. Application Data
(60) Provisional application No. 63/284,164, filed on Nov. 30, 2021.

(51) Int. Cl. C12N 5/0775; A61K 9/00; A61K 35/51; A61P 25/00

(52) U.S. Cl. CPC C12N 5/0665; A61K 9/0019; A61K 9/0048; A61K 35/51; A61P 25/00; C12N 2501/22; C12N 2501/2304; C12N 2506/1353; C12N 2510/00

(57) ABSTRACT

The present disclosure relates to engineered cells and uses thereof for treating neurological disorders.

### CROSS-REFERENCE TO RELATED APPLICATIONS

This application claims the benefit of U.S. Provisional Application No. 63/284,164, filed Nov. 30, 2021, which is expressly incorporated herein by reference in its entirety.

### INCORPORATION BY REFERENCE OF SEQUENCE LISTING

The content of the XML file named “103361-166WO1.xml,” which was created on Nov. 30, 2022, and is 3,711 bytes in size, is hereby incorporated by reference in its entirety.

### FIELD

The present disclosure relates to engineered cells and uses thereof.

### BACKGROUND

Central nervous system (CNS) injury secondary to trauma, neurodegenerative disease (such as Alzheimer's Disease, Parkinson's Disease and amyotrophic lateral sclerosis), glaucoma, autoimmune inflammation (such as in multiple sclerosis and neuromyelitis optica), or stroke, can result in permanent symptoms and disability, including visual loss, double vision, swallowing difficulties, speech impairment, weakness, spasticity, numbness, paresthesias, vertigo, gait instability, limb incoordination, tremor, neuropathic pain, and dementia. The risk of chronic neurological deficits in individuals with CNS damage is heightened by the inability of CNS neurons to replicate, and the limited capacity of severed axons to regrow in the optic nerve, brain and spinal cord. There is a dire need for new therapies that overcome intrinsic barriers to regeneration within the CNS, and mitigate, or even reverse, neuronal and axonal damage. What is needed are novel compositions and methods for improving neuronal survival and/or axon regeneration, and treating neurological disorders and traumatic injury.

### SUMMARY

In some aspects, disclosed herein is an engineered cell, wherein the engineered cell has a phenotype of CD33+. In some examples, the primary or engineered cell has a phenotype of CD33+CD34+ or CD33+CD34dim.

In some embodiments, the engineered cell is a primary or engineered human umbilical cord blood (HUCB) cell.

In some embodiments, the engineered cell is derived from a bone marrow cell. In some embodiments, the bone marrow cell is contacted with IL-4 and/or granulocyte colony-stimulating factor (G-CSF) to create the engineered cell. In some embodiments, the bone marrow cell is contacted with IL-4 and/or G-CSF in vitro or ex vivo to create the engineered cell.

In some embodiments, the engineered cell is derived from a stem cell obtained from a human, wherein the human was previously administered with G-CSF. the stem cell is contacted with IL-4 and/or G-CSF to create the engineered cell. In some embodiments, the stem cell is contacted with IL-4 and/or G-CSF in vitro or ex vivo to create the engineered cell.

In some embodiments, the engineered cell is enhanced in the expression of a neuroprotective molecule or growth factor as compared to a reference control. In some embodiments, the neuroprotective molecule is selected from the group consisting of nerve growth factor β (NGF β), brain derived growth factor (BDGF), ciliary neurotrophic factor receptor (CNTF), glial derived neurotrophic factor (GDNF), transforming growth factor-α (TGFα), transforming growth factor-β (TGFβ), interleukin-4 (IL-4), heparin-binding epidermal growth factor (HB-EGF), platelet-derived growth factor (PDGF), fibroblast growth factors (FGFs), granulocyte-colony stimulating factor (G-CSF), insulin-like growth factor 1 (IGF-1) and insulin-like growth factor 2 (IGF-2).

In some embodiments, the engineered cell is enhanced in the expression of a chemotactic receptor as compared to a reference control. In some embodiments, the chemotactic receptor comprises CCR2. In some embodiments, the chemotactic receptor comprises CCR3, CXCR2, or CXCR3.

In some embodiments, the engineered cell is enhanced in the expression of an anti-apoptotic molecule as compared to a reference control. In some embodiments, the anti-apoptotic molecule comprises BCL2L2.

Also disclosed herein is a composition comprising the cell of any preceding aspects.

In some embodiments, the composition further comprises a viral vector. In some embodiments, the viral vector comprises a polynucleotide sequence encoding IL-4 and/or a lateral olfactory tract usher substance (LOTUS) protein. In some embodiments, the viral vector is a lentiviral vector or an Adeno-associated virus (AAV) vector.

In some embodiments, the composition further comprises a small interfering RNA (siRNA), a short hairpin RNA (shRNA), or a CRISPR-Cas9 system. In some embodiments, the siRNA, the shRNA, or the CRISPR-Cas9 system comprises a polynucleotide sequence that targets phosphatase and tensin homolog (PTEN).

In some embodiments, the composition comprises soluble factors or vesicles (e.g., microvesicles) produced by cells. In some embodiments, the soluble products of HUCB cells are packaged in a pharmaceutically acceptable carrier.

In some aspects, disclosed herein is a method of treating a neurological disorder in a subject, comprising administering to the subject a therapeutically effective amount of the engineered cell or cell line of any preceding aspect or the composition of any preceding aspect.

In some aspects, disclosed herein is a method of treating a neurological disorder in a subject, comprising administering to the subject a therapeutically effective amount of the products or vesicles of the engineered cells or cell lines of any preceding aspect or the composition of any preceding aspect.

In some embodiments, the method further comprises administering to the subject a therapeutically effective amount of a viral vector. In some embodiments, the viral vector comprises a polynucleotide sequence encoding IL-4 and/or lateral olfactory tract usher substance (LOTUS) protein. In some embodiments, the viral vector is a lentiviral vector or an Adeno-associated virus (AAV) vector.

In some embodiments, the method further comprises administering to the subject a therapeutically effective amount of a small interfering RNA (siRNA), a short hairpin RNA (shRNA), or a CRISPR-Cas9 system. In some embodiments, the siRNA, the shRNA, or the CRISPR-Cas9 system comprises a polynucleotide sequence that targets phosphatase and tensin homolog (PTEN).

In some embodiments, the neurological disorder is an optic nerve or retinal disorder or injury. In some embodiments, the method improves the survival of a retinal ganglion cell and/or the regeneration of retinal ganglion cell axons. In some embodiments, the composition or the engineered cell is administered intraocularly or intravitreally.

In some embodiments, the neurological disorder is a spinal cord disorder or injury. In some embodiments, the method improves the survival of spinal cord neurons and/or the regeneration of spinal cord axons. In some embodiments, the composition or the engineered cell is administered intrathecally, intraventricularly, in the dorsal root ganglion, intravenously, or intraperitoneally.

In some embodiments, the neurological disorder is a brain disorder. In some embodiments, the method improves the survival of cerebral neurons and/or the regeneration of cerebral axons. In some embodiments, the composition or the engineered cell is administered intracranially, intraventricularly, intrathecally, intravenously or intraperitoneally.

In some embodiments, the primary or engineered cell is expanded in vitro in a cell culture medium prior to administration. In some embodiments, the medium comprises recombinant human stem cell factor, thrombopoietin, G-CSF, IL-4, TGF-β, and/or GM-CSF. In some embodiments, the engineered cell is expanded for about 7 to about 14 days.

In some embodiments, the primary or engineered cell is cultured with a dectin-1 ligand, a dectin-1 small molecule agonist, or an agonistic antibody of dectin-1.

Also disclosed herein is a method of improving the survival of a nerve cell, and/or the regeneration of nerve cell fibers (i.e., axons), in a subject, comprising administering to the subject a therapeutically effective amount of the CD33+ cell or cell line of any preceding aspect or the composition of any preceding aspect.

In some embodiments, the nerve cell is a retinal ganglion cell.

In some embodiments, the nerve cell is a spinal cord neuron.

In some embodiments, the nerve cell is a cortical or cerebral neuron.

In some embodiments, the method further comprises administering to the subject a therapeutically effective amount of a viral vector. In some embodiments, the viral vector comprises a polynucleotide sequence encoding IL-4 and/or lateral olfactory tract usher substance (LOTUS) protein. In some embodiments, the viral vector is a lentiviral vector or an Adeno-associated virus (AAV) vector.

In some embodiments, the method further comprises administering to the subject a therapeutically effective amount of a small interfering RNA (siRNA), a short hairpin RNA (shRNA), or a CRISPR-Cas9 system. In some embodiments, the siRNA, the shRNA, or the CRISPR-Cas9 system comprises a polynucleotide sequence that targets phosphatase and tensin homolog (PTEN).

In some embodiments, the subject has an optic nerve disorder.

In some embodiments, the composition or the cell is administered intraocularly or intravitreally.

In some embodiments, the nerve cell is a spinal cord neuron.

In some embodiments, the composition or the engineered cell is administered intrathecally, intraventricularly, in the dorsal root ganglion, intravenously, or intraperitoneally.

In some embodiments, the nerve cell is a cortical or cerebral neuron.

In some embodiments, the composition or the engineered cell is administered intracranially, intraventricularly, intrathecally, intravenously or intraperitoneally.

In some embodiments, the primary or engineered cell is expanded in vitro in a cell culture medium prior to administration. In some embodiments, the medium comprises recombinant human stem cell factor, thrombopoietin, G-CSF, IL-4, TGF-β, and/or GM-CSF. In some embodiments, the engineered cell is expanded for about 7 to about 14 days.

In some embodiments, the primary or engineered cell is cultured with a dectin-1 ligand, or a dectin-1 small molecule agonist or agonistic antibody.

### DESCRIPTION OF DRAWINGS

The accompanying figures, which are incorporated in and constitute a part of this specification, illustrate several aspects described below.

FIGS. 1A-1E show that IL-4 polarized murine Ly6G^{low} cells stimulate ON axon regrowth. (FIGS. 1A-1C) Bone marrow (BM) cells were flushed from the femurs of naïve C57BL/6 mice. Ly6G^{low} myeloid cells were isolated and cultured for 24 h in media alone or with recombinant IL-4 and/or G-CSF. FIG. 1A shows Arg-1 transcript levels, measured by qPCR, and normalized to β-actin. In FIG. 1B polarized or unpolarized Ly6G^{low} BM cells were washed, resuspended in fresh media, and co-cultured with explanted neonatal retinal ganglion cells (RGC). Neurite outgrowth was measured 24 h later. ****p<0.00001 vs. media; ^{#####}p<0.00001 vs. unpolarized cells. In FIG. 1C, IL-4/G-CSF polarized or unpolarized Ly6G^{low} BM cells were harvested, washed and adoptively transferred into the vitreous of syngeneic C57BL/6 mice on the day of ON crush injury and 2 days later (3×10⁵ cells/eye/dose). Optic nerves were harvested 2 weeks post-injury and regenerating axons quantified in longitudinal sections at serial distances from the crush site by an investigator blinded to experimental groups (n=8-12 nerves/group; ^{#}p<0.01, ^{##}p<0.001 vs unpolarized cell transfer; ***p<0.0001 vs. PBS). (FIGS. 1D and 1E) AAV9-IL-4 or AAV9 empty (AAV-Ctrl) vectors were injected i.o. into naïve mice. In FIG. 1D, vitreal fluid was collected 2 weeks later and IL-4 protein levels measured via sandwich ELISA. NT, not transduced. FIG. 1E shows that, 1 week following AAV9 inoculation, mice were subjected to ON crush injury and i.o. injection with either zymosan or PBS. The density of regenerating ON axons was quantified 2 weeks post-injury. (n=8-16 nerves/group; *p<0.01, **p<0.001 compared with the PBS+AAV-IL-4 group; ^{#}p.01, ^{##}p<0.001 compared with the i.o. zymosan+AAV-Ctrl group).

FIGS. 2A-2E show that CD33+ HUCB cells induce retinal ganglion cell (RGC) axon regeneration in vivo and in vitro. FIGS. 2A-2D show experiments using CD33⁺ and CD33⁻ cells that were isolated from the mononuclear fraction of human umbilical cord blood. (FIG. 2A) HUCB subsets obtained from 3 individual patients (designated P1, P2, and P3, respectively) were cultured separately in the presence of explanted neonatal murine RGC. Neurite outgrowth was measured 24 h later. ***p<0.0001. FIG. 2B shows representative images of human iPSC-derived RGC following culture with CD33 (left panel) or CD33⁺ (right) HUCB cells. In FIG. 2C, conditioned media (CM) was harvested from 24 h cultures of CD33⁺ HUCB cells obtained from patient P6 or P7, respectively. CM or unconditioned (control) media was added to cultures of human iPSC-derived RGC. Neurite outgrowth was measured 48 h later. ***p<0.001. In FIG. 2D, CD33⁺ HUCB myeloid cells, isolated from patients P3, P4 and P5, respectively, were independently injected into the vitreous fluid of different groups of RAG1−/− mice on the day of ON crush injury and 2 days later (3×10⁵ cells/eye/dose). Other groups of RAG1−/mice were injected i.o. with PBS as a negative control, or HL60 cells as a positive control, on days 0 and 2 post-crush injury. Regenerating axons were quantified in optic nerve sections, prepared 2 weeks post-injury. FIG. 2E shows representative ON sections from mice with ONC injury that were injected with CD33⁺ HUCB cells (lower panel) or PBS (upper panel).

FIGS. 3A-3D show that CD33⁺CD34⁺ HUCB cell lines have optic nerve (ON)-regenerative properties. In FIGS. 3A-3C, CD34⁺ and CD34⁻ myeloid cells were FACS sorted from CD33⁺ HUCB mononuclear cells and expanded with growth factors in vitro for 7-14 days. Cell lines derived from individual patients were cultured with neonatal murine retinal ganglion cells (RGCs) (FIG. 3A) or human iPSC-derived RGC (FIG. 3B). The bar graphs show results obtained with cell lines derived from individual patients, following 7d (FIGS. 3A, 3B) or 14 d of culture (FIG. 3B). Data in (FIG. 3A) are representative of 2 experiments that were performed with 2 independently derived cell lines and produced similar results. In FIG. 3C, RAG1−/−mice were injected i.o. with CD34 HUCB myeloid cells (3×10⁵ cells/eye/dose), HL60 cells (cells (3×10⁵ cells/eye/dose; positive control) or PBS, on days 0 and 2 post-crush injury. Regenerating axons were quantified in optic nerve sections, prepared 2 weeks post-injury. Data shown are representative of 3 independent experiments with cell lines from 3 separate donors (n=8-12 nerves/group). FIG. 3D shows representative ON sections from mice with ONC injury that were injected i.o. with PBS (upper panel) or CD34⁺CD33⁺ HUCB cells (lower panel). The broken lines indicate the crush site. *p<0.01, **p<0.001, ***p<0.0001 vs. CD34⁻ or media (FIGS. 3A, 3B) or PBS (FIG. 3C).

FIGS. 4A-4B show that LV vectors transduce HUCB cells at with a high degree of efficiency. CD33⁺CD34⁺ HUCB cells were incubated for 72 h with lentivirus (LV) vectors, either carrying the NGF gene in tandem with a GFP reporter (LV-GFP) or the GFP reporter only (LV-Ctrl). 293 cells were transduced in parallel as a positive control. FIG. 4A shows flow cytometric analysis of GFP expression, gating on CD33⁺CD34⁺ HUCB cells that were not transduced (blue) or transduced with LV-GFP (red). FIG. 4B shows NGFβ levels that were measured in supernatants, harvested from the cultures indicated, by sandwich ELISA.

FIGS. 5A-5D show maps of lentiviral vectors. FIG. 5A shows the control lentiviral vector map. There is no insert cloned into the downstream of EF1α promoter. As a result, this lentiviral only expresses the eGFP (green fluorescent protein). FIG. 5B shows the experimental lentiviral vector map. The human NGFbeta (new growth factor beta_NM_002506.2) was cloned into the downstream of EF1α promoter. As a result, this lentiviral vector expresses both human NGF and GFP. FIG. 5C shows the control lentiviral vector map. There is no insert cloned into the downstream of PGK promoter. As a result, this lentiviral only expresses the GFP. FIG. 5D shows the experimental lentiviral vector map. The human NGF beta (new growth factor beta_NM_002506.2) was cloned into the downstream of PGK promoter. As a result, this lentiviral vector expresses both human NGF and GFP.

FIGS. 6A-6C show that CD34 Human bone marrow myeloid precursor cells can be polarized to a neuroregenerative phenotype that stimulates axon outgrowth. FIG. 6A shows human cortical neuron axon length quantification after 24 hours of culture with human CD34⁺ bone marrow cells that have either been polarized with IL-4, GSCF, or IL-4 and GCSF for 24 hours compared to human cortical neurons cultured with unpolarized CD34⁺ cells or neurons cultured without human CD34⁺ bone marrow cells (Media) or with 100 ng of nerve growth factor (NGF). IL-4+GCSF polarized CD34′ bone marrow cells stimulate significantly more axon outgrowth compared to unpolarized CD34⁺ cells, neurons alone, or neurons with NGF (n=150 neurons counted per condition, ***p<0.001, **** p<0.0001). FIG. 6B shows representative images of human cortical neurons in each cultured condition. FIG. 6C shows human cortical neuron axon length quantification after 24 hours of culture with multiple donors of human CD34⁺ bone marrow cells that have either been polarized with IL-4, GCSF, or IL-4 and GCSF for 24 hours compared to human cortical neurons cultured with unpolarized CD34⁺ cells or neurons cultured without human CD34⁺ bone marrow cells (Media) or with 100 ng of nerve growth factor (NGF). IL-4+GCSF polarized CD34⁺ bone marrow cells stimulate significantly more axon outgrowth compared to unpolarized CD34⁺ cells, neurons alone, or neurons with NGF (each dot represents an individual culture with a unique donor, (n=6 donors and n=150 neurons counted per condition, ***p<0.001).

FIGS. 7A-7D show that human CD33⁺ and CD33⁺CD34⁺ myeloid cells isolated from human cord blood stimulate axon regeneration and neuroprotection. (FIG. 7A) HuBC CD33⁺ purified cells from 3 separate donors were placed in culture with mouse dorsal root ganglion (DRG) neurons for 20 hours, then quantified by immunohistochemistry for their ability to stimulate neurite outgrowth. Purified CD33+ HuBCs from all 3 donors stimulated neurite outgrowth compared to DRGs without cells (****p<0.0001, n=500 DRGs/group). (FIG. 7B) HuBC CD33⁺ CD34⁺ purified cells were cultured for 7 days for expansion then placed in co-culture with mouse DRGs stimulate neurite outgrowth compared to CD33− cells from culture. CD33+ CD34+ HuBCs stimulated DRG neurite outgrowth compared to DRGs cultured with CD34+ CD33− HuBCs (****p<0.0001, n=500 DRGs/group). (FIG. 7C) HuBC CD33⁺ purified cells placed in culture with iPSC derived human spinal motor neurons for 24 hours of co-culture together. The number of viable iPSC derived human spinal motor neurons with neurite growth was quantified by immunohistochemistry. iPSC derived human spinal motor neurons co-cultured with CD33+ HuBCs had more viable neurons with growing neurites compared to CD33− cells or neurons alone without cells in culture(*p<0.01). (FIG. 7D) Quantification of dorsal column axon regeneration or retraction 8 weeks after dorsal column transection and injection of either CD33+ or CD33− HuBCs into the sciatic nerve of the mouse. RAG1 KO mice were subjected to dorsal column lesion immediately followed by injection of either PBS, CD33⁺ or CD33− HuBCs into the sciatic nerve, Conditioning injury of the sciatic nerve was used as a positive control. 7 weeks after transection and injection of cells or PBS into the sciatic nerve, mice were injected with fluorescently conjugated dextran that was allowed to uptake along the dorsal column axons occurred for one week. Mice were sacrificed, spinal cords were harvested and cleared using Visikol clearing solution followed by imaging of the spinal cord by confocal microscopy for visualization of the spinal cord lesion and fluorescently labeled axons. Dorsal column axon regeneration across the spinal cord lesion towards the brain or retraction away from the lesion was measured by Imaris on 3D confocal images (n=6 spinal cords/group). HuCB CD33⁺ cells significantly stimulate axon regeneration across the spinal column lesion compared to CD33-HuBC or PBS treated mice (*p<0.01, **p<0.001).

FIGS. 8A-8E shows that extracellular vesicles (EVs) from CD33⁺CD34⁺ cells are neuroprotective and stimulate axon regeneration. (FIG. 8A) Spectrodyne quantification of EVs purified from cultured human cord blood (HuCB) purified CD33⁺CD34⁺ cells. (FIG. 8B) Dorsal root ganglion (DRG) neurite length quantification after 24 hours of culture exposure to increasing concentrations of HuCB CD33⁺CD34⁺ purified EVs shows that EVs stimulate neurite outgrowth in vitro in a dose dependent process. (FIG. 8C) Representative optic nerve regeneration with fluorescently labeled cholera toxin B tracer intra-ocular (i.o.) injection from RAG1 KO mice 2 weeks after optic nerve crush (ONC) and intraocular (i.o.) injection with either PBS or HuBC CD33⁺CD34⁺ EVs at 1×10¹⁰ EVs/eye at the time of crush and 3 days after crush. (FIG. 8D) Quantification of RGC axon regeneration 2 weeks after ONC and either i.o. PBS or EV injection (n=6 nerves/group). HuCB CD33⁺CD34⁺ EVs significantly stimulate axon regeneration (*p<0.01). (FIG. 8E) Quantification of viable RGC neurons/mm of retina two weeks after ONC and i.o. PBS or i.o. CD33′CD34⁺ EV injections. HuCB CD33⁺CD34⁺ EVs rescue RGCs from cell death compared to PBS injection (p<0.0001).

FIGS. 9A-9C show that extracellular vesicles (EVs) from CD33⁺ cells stimulate axon regeneration. (FIG. 9A) Dorsal root ganglion (DRG) neurite length quantification after 24 hours of culture exposure to two different donors of HuCB CD33⁺ purified EVs at a concentration of 1×10⁸ EVs from each donor shows that EVs stimulate neurite outgrowth in vitro from different donors. CD33⁺ HuCB EVs are generated by placing purified populations of CD33⁺ cells from donated human umbilical cord blood into culture for 24 hours and isolating EVs from the CD33⁺ cell culture. (FIG. 9B) Representative optic nerve regeneration with fluorescently labeled cholera toxin B tracer intra-ocular (i.o.) injection from RAG1 KO mice 2 weeks after optic nerve crush (ONC) and intraocular (i.o.) injection with either PBS or HuBC CD33⁺ EVs at 1×10⁸ EVs/eye at the time of crush and 3 days after crush. (FIG. 9C) Quantification of RGC axon regeneration 2 weeks after ONC and either i.o. PBS or EV injection (n=6 nerves/group). HuCB CD33⁺ EVs significantly stimulate axon regeneration (*p<0.01).

### DETAILED DESCRIPTION

Disclosed herein are primary cells, engineered cells, and extracellular vesicles and uses thereof for improving survival of neurons, regeneration of axons, and treating neurological disorders.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

The term “about” as used herein when referring to a measurable value such as an amount, a percentage, and the like, is meant to encompass variations of ±20%, ±10%, ±5%, or 1% from the measurable value.

“Administration” to a subject or “administering” includes any route of introducing or delivering to a subject an agent. Administration can be carried out by any suitable route, including oral, intravenous, intraperitoneal, intranasal, inhalation, intravitreal, intraocular and the like. Administration includes self-administration and the administration by another.

The term “biocompatible” generally refers to a material and any metabolites or degradation products thereof that are generally non-toxic to the recipient and do not cause significant adverse effects to the subject.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203.

A “control” is an alternative subject or sample used in an experiment for comparison purposes. A control can be “positive” or “negative.”

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “microvesicles”, as used herein, refers to a cell-derived membranous vesicle. They refer to extracellular vesicles, which are generally of between 30 and 1000 nm, for example in the range of between 30 and 200 nm or 50 and 100 nm in size. In some aspects, the extracellular vesicles can be in the range of 20 nm-300 nm in size, for example 30 nm-250 nm in size, for example 50 nm-200 nm in size. In some aspects, the extracellular vesicles are defined by a lipidic bilayer membrane. Extracellular vesicles are released from most cell types and can be found in many biological fluids. The extracellular vesicles used herein is derived from the engineered cells disclosed herein. In some embodiments, the extracellular vesicles comprise one or more biomarkers selected from the group consisting of CD9, CD63, and CD81.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “increased”, “increase”, or “enhanced” as used herein generally means an increase by a statically significant amount: for example, “increased” or “enhanced” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inhibitors” of expression or of activity are used to refer to inhibitory, activating, or modulating molecules, respectively, identified using in vitro and in vivo assays for expression or activity of a described target protein, e.g., antagonists and their homologs and mimetics. Inhibitors are agents that, e.g., inhibit expression or bind to, partially or totally block stimulation or activity, decrease, prevent, delay activation, inactivate, desensitize, or down regulate the activity of the described target protein, e.g., antagonists. Samples or assays comprising described target protein that are treated with a potential inhibitor are compared to control samples without the inhibitor to examine the extent of effect. Control samples (untreated with inhibitors) are assigned a relative activity value of 100%. Inhibition of a described target protein is achieved when the activity value relative to the control is about 80%, optionally 50% or 25, 10%, 5% or 1%.

As used herein, the term “level” refers to the amount of a target molecule in a sample, e.g., a sample from a subject. The amount of the molecule can be determined by any method known in the art and will depend in part on the nature of the molecule (i.e., gene, mRNA, cDNA, protein, enzyme, etc.). The art is familiar with quantification methods for nucleotides (e.g., genes, cDNA, mRNA, etc.) as well as proteins, polypeptides, enzymes, etc. It is understood that the amount or level of a molecule in a sample need not be determined in absolute terms, but can be determined in relative terms (e.g., when compared to a control or a sham or an untreated sample).

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “nucleic acid” as used herein means a polymer composed of nucleotides, e.g., deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides. (Used together with “polynucleotide” and “polypeptide”.)

The term “polypeptide” refers to a compound made up of a single chain of D- or L-amino acids or a mixture of D- and L-amino acids joined by peptide bonds.

The term “polynucleotide” refers to a single or double stranded polymer composed of nucleotide monomers.

As used herein, “pluripotent cells” means a population of cells capable of differentiating into all three germ layers and becoming any cell type in the body.

As used herein, “multipotent” cells are more differentiated than pluripotent cells, but are not permanently committed to a specific cell type. Pluripotent cells therefore have a higher potency than multipotent cells.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium, and/or nonionic surfactants such as TWEEN™ (ICI, Inc.; Bridgewater, New Jersey), polyethylene glycol (PEG), and PLURONICS™ (BASF; Florham Park, NJ).

The term “polymer” as used herein refers to a relatively high molecular weight organic compound, natural or synthetic, whose structure can be represented by a repeated small unit, the monomer. Synthetic polymers are typically formed by addition or condensation polymerization of monomers. The polymer is suitable for use in the body of a subject, i.e. is biologically inert and physiologically acceptable, non-toxic, and is biodegradable in the environment of use, i.e. can be resorbed by the body. The term “polymer” encompasses all forms of polymers including, but not limited to, natural polymers, synthetic polymers, homopolymers, heteropolymers or copolymers, addition polymers, etc.

The term “copolymer” as used herein refers to a polymer formed from two or more different repeating units (monomer residues). Copolymer compasses all forms copolymers including, but not limited to block polymers, random copolymers, alternating copolymers, or graft copolymers. A “block copolymer” is a polymer formed from multiple sequences or blocks of the same monomer alternating in series with different monomer blocks. Block copolymers are classified according to the number of blocks they contain and how the blocks are arranged.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

As used herein, a “target”, “target molecule”, or “target cell” refers to a biomolecule or a cell that can be the focus of a therapeutic drug strategy, diagnostic assay, or a combination thereof, sometimes referred to as a theranostic. Therefore, a target can include, without limitation, many organic molecules that can be produced by a living organism or synthesized, for example, a protein or portion thereof, a peptide, a polysaccharide, an oligosaccharide, a sugar, a glycoprotein, a lipid, a phospholipid, a polynucleotide or portion thereof, an oligonucleotide, an aptamer, a nucleotide, a nucleoside, DNA, RNA, a DNA/RNA chimera, an antibody or fragment thereof, a receptor or a fragment thereof, a receptor ligand, a nucleic acid-protein fusion, a hapten, a nucleic acid, a virus or a portion thereof, an enzyme, a co-factor, a cytokine, a chemokine, as well as small molecules (e.g., a chemical compound), for example, primary metabolites, secondary metabolites, and other biological or chemical molecules that are capable of activating, inhibiting, or modulating a biochemical pathway or process, and/or any other affinity agent, among others.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g., a composition comprising the cells, microvesicles, or soluble factors disclosed herein) refers to an amount that is effective to achieve a desired therapeutic result. In some embodiments, a desired therapeutic result is the control of a neurological disorder. In some embodiments, a desired therapeutic result is the control of a neurological disorder, or a symptom of a neurological disorder. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

### Engineered Cells

In some aspects, disclosed herein is an engineered cell.

The term “engineered cell” as used herein refers to a cell modified by means of genetic engineering, or polarization with, for example, soluble factors and/or ligands, or isolated cells with characteristics differing from any naturally occurring cell. In some embodiments, the engineered cell is a HUCB cell. In some embodiments, the engineered HUCB cell is a CD33+ myeloid cell. In some embodiments, the engineered cell is a CD33+ cell derived from a bone marrow cell. In some embodiments, the engineered cell is derived from a stem cell obtained from a human, wherein the human was previously administered with G-CSF. The engineered cells disclosed herein can express CD33 at various level. In some examples, the cells are CD33+CD34+, CD33+CD34dim, or CD33dimCD34+. In some embodiments, the engineered cell has a phenotype of CD33+CD34+ or CD33+CD34dim. In some embodiments, the engineered cell is an isolated cell or plurality of isolated cells.

CD33 or Siglec-3 is a transmembrane receptor expressed on cells of myeloid lineage. In some embodiments, the CD33 polypeptide is that identified in one or more publicly available databases as follows: HGNC: 1659 NCBI, Entrez Gene: 945, Ensembl: ENSG00000105383, OMIM®: 159590, UniProtKB/Swiss-Prot: P20138.

CD34 is a transmembrane phosphoglycoprotein, first identified on hematopoietic stem and progenitor cells. In some embodiments, the CD34 polypeptide is that identified in one or more publicly available databases as follows: HGNC: 1662, NCBI Entrez Gene: 947, Ensembl: ENSG00000174059, OMIM®: 142230, UniProtKB/Swiss-Prot: P28906.

Accordingly, the CD33+CD34+ cells and the CD33+CD34dim cells described herein maintain multipotency. In some embodiments, the engineered CD33+ cells disclosed herein are derived from primary HUCB cells.

In some embodiments, the engineered CD33+ cells disclosed herein are derived from a bone marrow cell (e.g., a hematopoietic cell in bone marrow), human umbilical cord blood, or GCSF mobilized blood cells.

The engineered CD33+ cells disclosed herein can be created by contacting a bone marrow cell with IL-4 and/or granulocyte colony-stimulating factor (G-CSF). In some embodiments, the bone marrow cell is contacted with IL-4 and/or G-CSF in vitro or ex vivo to create the engineered cell. In some embodiments, the bone marrow cell is contacted with IL-4 and/or G-CSF in vitro or ex vivo for at least about 3, 4, 5, 6, 7, 8, 9, 10, 11, 12, 13, 14, 15, 16, 17, 18, 20, 22, 24, 26, 28, 30, 35, 40, 45, 50, 60, 70, 80, 90, 100, 110, or 120 hours to create the engineered cell.

In some embodiments, the engineered CD33+ cells disclosed herein are derived from a granulocyte colony-stimulating factor (G-CSF)-mobilized cell (e.g., a G-CSF-mobilized hematopoietic stem cell). G-CSF can be used to therapeutically mobilize hematopoietic stem cells (HSC) to the bloodstream in a subject for transplantation. Accordingly, in some embodiments, the engineered cell is derived from a stem cell obtained from a human, wherein the human was previously administered with G-CSF. In some embodiments, the G-CSF-mobilized stem cells obtained from the subject are then contacted with IL-4 and/or G-CSF to create the engineered cell.

In some embodiments, the engineered CD33+ cells disclosed herein are differentiated from a stem cell (including, for examples, a hematopoietic stem cell in bone marrow, an embryonic stem cells, a mesenchymal stem cells, or an induced pluripotent stem cells (iPSC)).

As used herein, “induced pluripotent stem cells” or “iPSC” are cells that are differentiated, somatic cells reprogrammed to pluripotency. The cells are substantially genetically identical to their respective differentiated somatic cell of origin and display characteristics similar to higher potency cells, such as ES cells. See, Yu J, et al., “Induced pluripotent stem cell lines derived from human somatic cells,” Science 318:1917-1920 (2007), incorporated herein by reference as if set forth in its entirety.

The skilled person is aware of various methods to obtain stem cell, (e.g., iPSC or embryonic stem cell), derived neurons. When stem cells are removed from differentiation suppression conditions and/or when grown in suspension aggregates, called embryoid bodies, spontaneous differentiation to cells of the three germ layers occurs. Methods for stem cell differentiation are known in the art. See, e.g., U.S. Pat. Nos. 10,696,947 and 9,395,354, incorporated by reference herein in their entireties.

In some embodiments, the engineered cell (e.g., the HUCB cell or CD33+ myeloid cell disclosed herein) is enhanced in the expression of a neuroprotective molecule or growth factor as compared to a reference control. In some embodiments, the neuroprotective molecule or growth factor is selected from the group consisting of nerve growth factor β (NGF β), brain derived growth factor (BDGF), ciliary neurotrophic factor receptor (CNTF), glial derived neurotrophic factor (GDNF), transforming growth factor-β (TGFβ), transforming growth factor-α (TGFα), interleukin-4 (IL-4), heparin-binding epidermal growth factor (HB-EGF), fibroblast growth factors (FGFs), platelet-derived growth factor (PDGF), granulocyte-colony stimulating factor (G-CSF), insulin-like growth factor 1 (IGF-1), hepatocyte Growth Factor (HGF), and insulin-like growth factor 2 (IGF-2).

In some embodiments, the engineered cell (e.g., the HUCB cell or CD33+ myeloid cell disclosed herein) is enhanced in the expression of a chemotactic receptor as compared to a reference control. In some embodiments, the chemotactic receptor comprises CCR2, CCR3, CXCR2 or CXCR3.

In some embodiments, the engineered cell (e.g., the HUCB cell or CD33+ myeloid cell disclosed herein) is enhanced in the expression of an anti-apoptotic molecule as compared to a reference control. In some embodiments, the anti-apoptotic molecule comprises BCL2L2, BCL-XL, MCL-1, BFL-1, BCL-W, or BCL2L10.

The term “reference control” refers to a level in detected in a subject in general or a study population.

In some aspects, disclosed herein is a composition comprising the engineered cell (e.g., the HUCB cell or CD33+ myeloid cell) disclosed herein. In some embodiments, the composition further comprises a vector. In some embodiments, the vector comprises a polynucleotide sequence encoding IL-4 and/or lateral olfactory tract usher substance (LOTUS) protein.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Viral vector” as disclosed herein means, in respect to a vehicle, any virus, virus-like particle, virion, viral particle, or pseudotyped virus that comprises a nucleic acid sequence that directs packaging of a nucleic acid sequence in the virus, virus-like particle, virion, viral particle, or pseudotyped virus. In some embodiments, the virus, virus-like particle, virion, viral particle, or pseudotyped virus is capable of transferring a vector (such as a nucleic acid vector) into and/or between host cells. In some embodiments, the virus, virus-like particle, virion, viral particle, or pseudotyped virus is capable of transferring a vector (such as a nucleic acid vector) into and/or between target cells, such as a hepatocyte in the liver of a subject. Importantly, in some embodiments, the virus, virus-like particle, virion, viral particle, or pseudotyped virus is capable of transporting into a nucleus of a target cell (e.g., a hepatocyte). The term “viral vector” is also meant to refer to those forms described more fully in U.S. Patent Application Publication No. 2018/0057839, which is incorporated herein by reference for all purposes. Suitable viral vectors include, e.g., adenoviruses, adeno-associated virus (AAV), vaccinia viruses, herpesviruses, baculoviruses and retroviruses, parvoviruses, and lentiviruses. In some embodiments, the viral vector is a lentiviral vector. Methods for gene delivery are known in the art. See, e.g., U.S. Pat. Nos. 5,399,346, 5,580,859, 5,589,466, incorporated by reference herein in their entireties.

In some embodiments, the viral vector is an adeno-associated virus (AAV) vector (e.g., AAV9). In some embodiments, the viral vector is a lentiviral vector.

Accordingly, in some embodiments, the viral vector polynucleotide sequence encoding IL-4 and/or lateral olfactory tract usher substance (LOTUS) protein. The viral vector can be used for increasing an expression level of IL-4 and/or LOTUS protein. In some embodiments, the AAV IL-4 and LOTUS is cloned the gene of interest under the control of a p-CMV promoter and placed in an AAV-9 serotype.

In some embodiments, the composition further comprises a small interfering RNA (siRNA), a short hairpin RNA (shRNA), or a CRISPR-Cas9 system for decreasing or silencing an expression level of a target protein.

The term “silencing” as used herein refers to suppression of expression of the (target) gene. It does not necessarily imply reduction of transcription, because gene silencing is believed to operate in at least some cases post-transcriptionally. The degree of gene silencing can be complete so as to abolish production of the encoded gene product (yielding a null phenotype), but more generally the gene expression is partially silenced, with some degree of expression remaining (yielding an intermediate phenotype). The term should not therefore be taken to require complete “silencing” of expression.

siRNA, also known as short interfering RNA or silencing RNA, is a class of double-stranded RNA molecules (for examples, 20-25 base pairs in length), which interferes with the expression of specific genes with complementary nucleotide sequences by degrading mRNA after transcription, preventing translation (Agrawal et al., Microbiol. Ivfol. Biol. Rev., 67(4): 657-668 (2003). shRNAs are artificial RNA molecules with a tight hairpin turn that can be used to silence target gene expression via RNAi.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. CRISPR systems are known in the art. See, e.g., U.S. Pat. No. 8,697,359, incorporated by reference herein in its entirety.

In some embodiments, the gene targeted by the siRNA, the shRNA, or the CRISPR-Cas9 system disrobed herein is phosphatase and tensin homolog (PTEN). Accordingly, in some embodiments, the siRNA, the shRNA, or the CRISPR-Cas9 system disclosed herein comprises a polynucleotide sequence that PTEN. In some embodiments, the shRNA sequence for targeting human PTEN comprises the sequences as follows:
(1)—Clone ID: V3SVHS02_6210945-mature antisense-TGAACATTGGAATAGTTTC (SEQ ID NO: 1);
(2)—Clone ID: V3SVHS02_7481841-mature antisense-TTGAACTGCTAGCCTCTGG (SEQ ID NO: 2);
(3)—Clone ID: V3SVHS02_7874244-mature antisense-ACAACAAGCAGTGACAGCG (SEQ ID NO: 3).

In some embodiments, the polypeptides or polynucleotides disclosed herein are those identified in publicly available database as follows: human nerve growth factor β (NGF β)-NM_002506.2, human brain derived growth factor (BDGF)-NM_001709, human ciliary neurotrophic factor receptor (CNTF), NM_000614.4, human glial derived neurotrophic factor (GDNF)-CR541923, human transforming growth factor-β (TGF β)-M60316.1, human transforming growth factor-α (TGF α)-NM_003236.4, human interleukin-4 (IL-4)-NM_000589.4, human heparin-binding epidermal growth factor (HB-EGF)-NM_001945.3, human fibroblast growth factors (FGFs)-M34187.1, human granulocyte-colony stimulating factor (G-CSF)-E01219.1, human insulin-like growth factor 1 (IGF-1)-M59316.1, Human insulin-like growth factor 2 (IGF-2)-NM_001111284.2, human CCR2-NM_001123041.3, human CCR3-NM_001837.4, human CXCR2-NM_001557.4, human CXCR3-NM_001504.2, human BCL2L2-BC021198.2, human BCL-XL-Z23115.1, human MCL-I-NM_021960.5, human BFL-1-U27467.1, human BCL-W-U59747.1, human BCL2L10-NM_001306168.1, and human LOTUS-NM_018058.7.

In some embodiments, the composition disclosed herein further comprises zymosan.

In some embodiments, the composition is formulated with a pharmaceutically acceptable carrier.

In some aspects, disclosed herein is a kit comprising a plurality of the engineered cells disclosed herein. In some aspects, disclosed herein is a kit comprising a plurality of CD33+ human umbilical cord blood (HUBC) cells. In some embodiments, the CD33+ HUCB cell has a phenotype of CD33+CD34+ or CD33+CD34dim. In some embodiments, the CD33+ HUCB cell is a primary HUCB cell. In some embodiments, the engineered cell is derived from a bone marrow cell or a G-CSF mobilized stem cell. In some aspects, disclosed herein is a kit comprising a plurality of the extracellular vesicles disclosed herein. In some embodiments, the extracellular vesicles are produced by the engineered cell disclosed herein (e.g., a CD33+ HUBC or a CD33+ engineered cell derived from a bone marrow cell or a G-CSF mobilized stem cell).

### Extracellular Vesicles

Provided herein, in some cases, are compositions comprising extracellular vesicles and methods of producing extracellular vesicles. The extracellular vesicles are produced by the engineered cell disclosed herein (e.g., a CD33+ HUBC or a CD33+ engineered cell derived from a bone marrow cell or a G-CSF mobilized stem cell). In some cases, the extracellular vesicles are any membrane-bound particle (e.g., a vesicle with a lipid bilayer). Often, the extracellular vesicles provided herein are secreted by a cell. In some instances, the extracellular vesicles are produced in vitro. In some instances, the extracellular vesicles are produced in vivo. In some instances, the extracellular vesicle is an exosome, a microvesicle, a retrovirus-like particle, an apoptotic body, an apoptosome, an oncosome, an exopher, an enveloped virus, an exomere, or other very large extracellular vesicle such as a large oncosome. In some cases, the extracellular vesicle is an exosome.

In some cases, the extracellular vesicles can have a diameter of about 10 nm to about 50,000 nm. In some cases, the extracellular vesicles can have a diameter of about 10 nm to about nm, about 10 nm to about 30 nm, about 10 nm to about 50 nm, about 10 nm to about 100 nm, about 10 nm to about 200 nm, about 10 nm to about 500 nm, about 10 nm to about 1,000 nm, about 10 nm to about 2,000 nm, about 10 nm to about 5,000 nm, about 10 nm to about 10,000 nm, about 10 nm to about 50,000 nm, about 20 nm to about 30 nm, about 20 nm to about 50 nm, about nm to about 100 nm, about 20 nm to about 200 nm, about 20 nm to about 500 nm, about 20 nm to about 1,000 nm, about 20 nm to about 2,000 nm, about 20 nm to about 5,000 nm, about 20 nm to about 10,000 nm, about 20 nm to about 50,000 nm, about 30 nm to about 50 nm, about 30 nm to about 100 nm, about 30 nm to about 200 nm, about 30 nm to about 500 nm, about 30 nm to about 1,000 nm, about 30 nm to about 2,000 nm, about 30 nm to about 5,000 nm, about 30 nm to about 10,000 nm, about 30 nm to about 50,000 nm, about 50 nm to about 100 nm, about 50 nm to about 200 nm, about 50 nm to about 500 nm, about 50 nm to about 1,000 nm, about 50 nm to about 2,000 nm, about 50 nm to about 5,000 nm, about 50 nm to about 10,000 nm, about 50 nm to about 50,000 nm, about 100 nm to about 200 nm, about 100 nm to about 500 nm, about 100 nm to about 1,000 nm, about 100 nm to about 2,000 nm, about 100 nm to about 5,000 nm, about 100 nm to about 10,000 nm, about 100 nm to about 50,000 nm, about 200 nm to about 500 nm, about 200 nm to about 1,000 nm, about 200 nm to about 2,000 nm, about 200 nm to about 5,000 nm, about 200 nm to about 10,000 nm, about 200 nm to about 50,000 nm, about 500 nm to about 1,000 nm, about 500 nm to about 2,000 nm, about 500 nm to about 5,000 nm, about 500 nm to about 10,000 nm, about 500 nm to about 50,000 nm, about 1,000 nm to about 2,000 nm, about 1,000 nm to about 5,000 nm, about 1,000 nm to about 10,000 nm, about 1,000 nm to about 50,000 nm, about 2,000 nm to about 5,000 nm, about 2,000 nm to about 10,000 nm, about 2,000 nm to about 50,000 nm, about 5,000 nm to about 10,000 nm, about 5,000 nm to about 50,000 nm, or about 10,000 nm to about 50,000 nm. In some cases, the extracellular vesicles have a diameter of about 10 nm, about 20 nm, about 30 nm, about 50 nm, about 100 nm, about 200 nm, about 500 nm, about 1,000 nm, about 2,000 nm, about 5,000 nm, about 10,000 nm, or about 50,000 nm. In some cases, the extracellular vesicles can have a diameter at least about 10 nm, about 20 nm, about 30 nm, about 50 nm, about 100 nm, about 200 nm, about 500 nm, about 1,000 nm, about 2,000 nm, about 5,000 nm, or about 10,000 nm. In some cases, the extracellular vesicles can have a diameter at most about 20 nm, about 30 nm, about 50 nm, about 100 nm, about 200 nm, about 500 nm, about 1,000 nm, about 2,000 nm, about 5,000 nm, about 10,000 nm, or about 50,000 nm.

In some cases, the extracellular vesicle comprises at least one targeting polypeptide. In some cases, the extracellular vesicle comprises at least one targeting polypeptide and at least one therapeutic.

### Methods of Treatment

The primary or engineered cells disclosed herein can improve neuronal survival/axon regeneration and treatment of neurological disorders. Accordingly, in some aspect, disclosed herein is a method of treating, preventing, and/or mitigating a neurological disorder, comprising administering to the subject a therapeutically effective amount of the engineered cell, or its products, disclosed herein. In some embodiments, the engineered cell is a HUCB cell. In some embodiments, the engineered HUCB cell is a CD33+ myeloid cell. In some embodiments, the engineered HUCB cell has a phenotype of CD33⁺CD34⁺ or CD33⁺CD34dim. In some embodiments, the engineered cell is derived from a bone marrow cell or a G-CSF mobilized cell.

In some embodiments, the engineered cell is enhanced in the expression of a neuroprotective molecule or growth factor as compared to a reference control. In some embodiments, the neuroprotective molecule is selected from the group consisting of nerve growth factor β (NGF β), brain derived growth factor (BDGF), ciliary neurotrophic factor receptor (CNTF), glial derived neurotrophic factor (GDNF), platelet-derived growth factor (PDGF), transforming growth factor-β (TGFβ), transforming growth factor-α (TGFα), interleukin-4 (IL-4), heparin-binding epidermal growth factor (HB-EGF), fibroblast growth factors (FGFs), granulocyte-colony stimulating factor (G-CSF) and insulin-like growth factor 1 (IGF-1) and insulin-like growth factor 2 (IGF-2).

In some embodiments, the engineered cell is enhanced in the expression of a chemotactic receptor as compared to a reference control. In some embodiments, the chemotactic receptor comprises CCR2, CCR3, CXCR2 or CXCR3.

In some embodiments, the engineered cell is enhanced in the expression of an anti-apoptotic molecule as compared to a reference control. In some embodiments, the anti-apoptotic molecule comprises BCL2L2, BCL-XL, MCL-1, BFL-1, BCL-W, or BCL2L10.

It should be understood and herein contemplated that additional gene editing tools can be administered to the subject in combination with cellular therapy to intervene target axon growth inhibitory pathways, promote neuron survival, and improve neuron regeneration.

Accordingly, in some aspects, disclosed herein is a method of treating, preventing, and/or mitigating a neurological disorder, comprising administering to the subject a therapeutically effective amount of the composition disclosed herein, wherein the composition comprises the engineered cell (e.g., the HUCB cells, cells derived from bone marrow cells or G-CSF mobilized cells) or its products disclosed herein (e.g., extracellular vesicles) and a vector. In some embodiments, the vector polynucleotide sequence encoding IL-4 and/or lateral olfactory tract usher substance (LOTUS) protein. In some embodiments, the vector is a viral vector (e.g., an AAV9 vector). In some embodiments, the composition further comprises a small interfering RNA (siRNA), a short hairpin RNA (shRNA), or a CRISPR-Cas9 system for decreasing or silencing an expression level of a target protein. In some embodiments, the si RNA, the shRNA, or the CRISPR-Cas9 system disclosed herein comprises a polynucleotide sequence that PTEN.

The gene editing tools of any preceding aspects can be administered to the subject prior to or after the administration of the primary or engineered cells or their products, or simultaneously with the engineered cells or their products. Accordingly, in some embodiments, the method disclosed herein further comprises administering to the subject a therapeutically effective amount of a small interfering RNA (siRNA), a short hairpin RNA (shRNA), or a CRISPR-Cas9 system. In some embodiments, the siRNA, the shRNA, or the CRISPR-Cas9 system disclosed herein comprises a polynucleotide sequence that PTEN. in some embodiments, the method disclosed herein further comprises administering to the subject a therapeutically effective amount of a viral vector. In some embodiments, the viral vector comprises a polynucleotide sequence encoding IL-4 and/or lateral olfactory tract usher substance (LOTUS) protein. In some embodiments, the method further comprises administering to the subject an effective amount of zymosan or other dectin-1 ligand, dectin-1 small molecule agonist, or a dectin-1 agonistic antibody. In some embodiments, the method further comprises administering to the subject an effective amount of a CXCR2 blockade (e.g., an antibody blocking CXCR2).

In some embodiments, the shRNA sequence for targeting human PTEN comprises the sequences as follows:
(1)—Clone ID: V3SVHS02_6210945-mature antisense-TGAACATTGGAATAGTTTC (SEQ ID NO: 1);
(2)—Clone ID: V3SVHS02_7481841-mature antisense-TTGAACTGCTAGCCTCTGG (SEQ ID NO: 2);
(3)—Clone ID: V3SVHS02_7874244-mature antisense-ACAACAAGCAGTGACAGCG (SEQ ID NO: 3).

In some embodiments, the polypeptides or polynucleotides disclosed herein are those identified in publicly available database as follows. human nerve growth factor β (NGF β)-NM_002506.2, human brain derived growth factor (BDGF)-NM_001709, human ciliary neurotrophic factor receptor (CNTF), NM_000614.4, human glial derived neurotrophic factor (GDNF)-CR541923, human transforming growth factor-β (TGF β)-M60316.1, human transforming growth factor-α (TGF α)-NM_003236.4, human interleukin-4 (IL-4)-NM_000589.4, human heparin-binding epidermal growth factor (HB-EGF)-NM_001945.3, human fibroblast growth factors (FGFs)-M34187.1, human granulocyte-colony stimulating factor (G-CSF)-E01219.1, human insulin-like growth factor 1 (IGF-1)-M59316.1, Human insulin-like growth factor 2 (IGF-2)-NM_001111284.2, human CCR2-NM_001123041.3, human CCR3-NM 001837.4, human CXCR2-NM 001557.4, human CXCR3-NM 001504.2, human BCL2L2-BC021198.2, human BCL-XL-Z23115.1, human MCL-1-NM_021960.5, human BFL-1-U27467.1, human BCL-W-U59747.1, human BCL2L10-NM_001306168.1, and human LOTUS-NM_018058.7.

As used herein, the term “neurological disorder” refers to a disease or disorder in the central nervous system (CNS, brain, and spinal cord), and includes, but is not limited to, neurodegenerative diseases and disorders, or central nervous system (CNS) injury secondary to trauma, neurodegenerative disease (such as Alzheimer's Disease, Parkinson's Disease and amyotrophic lateral sclerosis), glaucoma, autoimmune inflammation (such as in multiple sclerosis and neuromyelitis optica), or stroke. CNS injury can result in permanent symptoms and disability, including visual loss, double vision, swallowing difficulties, speech impairment, weakness, spasticity, numbness, paresthesias, vertigo, gait instability, limb incoordination, tremor, neuropathic pain, and dementia.

In some embodiments, the neurological disorder is neurodegenerative disease. As used herein, the term “neurodegenerative disease” refers to a varied assortment of central nervous system disorders characterized by loss of neural tissue and/or neural tissue function. In some embodiments, the neurological disorder is an optic nerve disorder or injury. In some embodiments, the composition or the engineered HUCB cell is administered intraocularly or intravitreally.

In some embodiments, the neurological disorder is a brain or spinal cord disorder or injury.

In some embodiments, the neurological disorder results from physical trauma to the optic nerve, brain, or spinal cord.

In some embodiments, the neurological disorder results from ischemic or hemorrhagic damage to the optic nerve, brain, and or spinal cord.

As used herein, the term “neurodegenerative disease” refers to a varied assortment of central nervous system disorders characterized by gradual and progressive loss of neural tissue and/or neural tissue function. A neurodegenerative disease is a class of neurological disorder or disease, and where the neurological disease is characterized by a gradual and progressive loss of neural tissue, and/or altered neurological function, typically reduced neurological function as a result of a gradual and progressive loss of neural tissue. Examples of neurodegenerative diseases include for example, but are not limited to, Alzheimer's disease (AD), Parkinson's disease (PD), Huntington's Disease, Amyotrophic Lateral Sclerosis (ALS, also termed Lou Gehrig's disease) and Multiple Sclerosis (MS), polyglutamine expansion disorders (e.g., HD, dentatorubropallidoluysian atrophy, Kennedy's disease (also referred to as spinobulbar muscular atrophy), spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple system atrophy, Pelizaeus-Merzbacher disease, Pick's disease, primary lateral sclerosis, Refsum's disease, Sandhoff disease, Schilder's disease, spinal cord injury, spinal muscular atrophy (SMA), Steele-Richardson-Olszewski disease, Tabes dorsalis, and the like. In some embodiments, the neurodegenerative disease is Alzheimer's disease.

“Alzheimer's Disease” as used herein refers to all form of dementia, identified as a degenerative and terminal cognitive disorder. The disease may be static, the result of a unique global brain injury, or progressive, resulting in long-term decline in cognitive function due to damage or disease in the body beyond what might be expected from normal aging. Particularly, it has been identified that Alzheimer's disease is caused by the accumulation of the beta-amyloid protein, or Aβ, and it has been known that the induction of the degeneration and death of nerve cells caused by the amyloid protein is involved in the mechanism of Alzheimer's disease.

It should be understood that a treatment of Alzheimer's Disease may be a treatment of one or more of memory loss, poor judgment leading to bad decisions, loss of spontaneity and sense of initiative, repeating questions, having difficulties to organize thoughts, mood and personality changes, and/or increased anxiety and/or aggression. Treatment can be indicated by one or more of mental status and neuropsychological testing indicating an improvement in memory and/or other thinking skills, and/or mitigation of loss memory, brain imaging (e.g., using magnetic resonance imaging (MRI), computerized tomography (CT), or positron emission tomography (PET)) indicating mitigation of brain shrinkage, amyloid deposits, and/or neurofibrillary tangles, and/or improvement in nutrient metabolism in brain.

In some embodiments, the engineered cell is expanded in vitro in a cell culture medium prior to administration. In some embodiments, the medium comprises recombinant human stem cell factor, thrombopoietin, G-CSF, IL-4, TGFβ, or GM-CSF or a combination thereof. In some embodiments, the engineered HUCB cell is expanded for at least 3 days (e.g., at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 22 days, at least 25 days, at least 30 days, or at least 60 days). In some embodiments, the engineered HUCB cell is expanded for about 5 to about 30 days, for about 5 days to 20 days, or for about 7 to about 14 days.

Also disclosed herein is a method of improving the survival of a nerve cell and/or regeneration of a nerve cell fiber in a subject, comprising administering to the subject a therapeutically effective amount of the cell/cell line or cell products disclosed herein or the composition disclosed herein. In some embodiments, the nerve cell is a retinal ganglion cell.

In some embodiments, the method further comprises administering to the subject a therapeutically effective amount of a viral vector. In some embodiments, the viral vector comprises a polynucleotide sequence encoding IL-4 and/or lateral olfactory tract usher substance (LOTUS) protein. In some embodiments, the viral vector is a lentiviral vector or a Adeno-associated virus (AAV) vector.

In some embodiments, the method further comprises administering to the subject a therapeutically effective amount of a small interfering RNA (siRNA), a short hairpin RNA (shRNA), or a CRISPR-Cas9 system. In some embodiments, the siRNA, the shRNA, or the CRISPR-Cas9 system comprises a polynucleotide sequence that targets phosphatase and tensin homolog (PTEN). In some embodiments, the method further comprises administering to the subject an effective amount of zymosan or other dectin-1 ligand, or a dectin-1 small molecule agonist or an agonistic antibody of dectin-1. In some embodiments, the method further comprises administering to the subject an effective amount of a CXCR2 blockade (e.g., an antibody blocking CXCR2).

In some embodiments, the subject has an optic nerve disorder. In some embodiments, the composition or the cell is administered intraocularly or intravitreally.

In some embodiments, the engineered cell is expanded in vitro in a cell culture medium prior to administration. In some embodiments, the medium comprises recombinant human stem cell factor, thrombopoietin, G-CSF, IL-4, TGF-β, and/or GM-CSF or a combination thereof. In some embodiments, the engineered HUCB cell is expanded for at least 3 days (e.g., at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 22 days, at least 25 days, at least 30 days, or at least 60 days). In some embodiments, the engineered cell is expanded for about 5 to about 30 days, for about 5 days to 20 days, or for about 7 to about 14 days.

Described herein are methods of treating a disease in a subject by administering a therapeutic effective amount of the composition or pharmaceutical composition comprising the extracellular vesicle described herein. In some cases, the extracellular vesicle comprises the at least one targeting polypeptide and at least one therapeutic.

Dosing frequency for the therapeutic agent disclosed herein, includes, but is not limited to, at least once every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks, once a week, twice a week, three times a week, four times a week, five times a week, six times a week, or daily. In some embodiments, the interval between each administration is less than about 4 months, less than about 3 months, less than about 2 months, less than about a month, less than about 3 weeks, less than about 2 weeks, or less than less than about a week, such as less than about any of 6, 5, 4, 3, 2, or 1 day. In some embodiments, the dosing frequency for composition includes, but is not limited to, at least once a day, twice a day, or three times a day. In some embodiments, the interval between each administration is less than about 48 hours, 36 hours, 24 hours, 22 hours, 20 hours, 18 hours, 16 hours, 14 hours, 12 hours, 10 hours, 9 hours, 8 hours, or 7 hours. In some embodiments, the interval between each administration is less than about 24 hours, 22 hours, 20 hours, 18 hours, 16 hours, 14 hours, 12 hours, 10 hours, 9 hours, 8 hours, 7 hours, or 6 hours. In some embodiments, the interval between each administration is constant. For example, the administration can be carried out daily, every two days, every three days, every four days, every five days, or weekly. Administration can also be continuous and adjusted to maintaining a level of the compound within any desired and specified range.

Also disclosed herein is a method of treating a neurological disorder (e.g., an optical nerve disorder) in a subject, comprising administering to the subject a therapeutically effective amount of Ly6GlowCD14+ neutrophil and a viral vector that comprises a polynucleotide encoding a IL-4 protein. In some embodiments, the viral vector is a lentiviral vector or a Adeno-associated virus (AAV) vector. In some embodiments, the method improves the survival of a retinal ganglion cell or regeneration of retinal ganglion cell axons. In some embodiments, the Ly6GlowCD14+ neutrophil is administered intraocularly or intravitreally. In some embodiments, the viral vector is administered intraocularly or intravitreally.

Also disclosed herein is a method of improving the survival of retinal ganglion cells, and/or regeneration of retinal ganglion cell axons in a subject, comprising administering to the subject a therapeutically effective amount of a therapeutically effective amount of Ly6GlowCD14+ neutrophil and a viral vector that comprises a polynucleotide encoding an IL-4 protein. In some embodiments, the viral vector is a lentiviral vector or a Adeno-associated virus (AAV) vector. In some embodiments, the method improves the regeneration of a retinal ganglion cell. In some embodiments, the Ly6GIowCD14+ neutrophil is administered intraocularly or intravitreally.

In some embodiments, the method further comprises administering to the subject an effective amount of zymosan, or other dectin-1 ligand, or an anti-dectin-1 agonistic antibody. In some embodiments, the method further comprises administering to the subject an effective amount of CXCR2 blockade (e.g., an antibody blocking CXCR2).

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. CD33⁺ Human Umbilical Cord Blood (HUCB) Myeloid Cells and Short Term CD33⁺CD34⁺ HUCB Cell Lines Stimulate the Outgrowth of Adult Retinal Ganglion Cell (RGC) Axons

One of the functions of the immune system is to repair tissues that are collaterally damaged in the wake of infection, trauma, or other insults. Although this has been most clearly demonstrated in animal models of cutaneous wounds and myocardial ischemia, immune-mediated healing has also been observed in the CNS and in the eye. The current example identifies and characterizes neuroprotective, neuroregenerative human myeloid cells, and explore their use as a cellular therapy.

Human umbilical cord blood (HUCB) is a potential source of immature hematopoietic cells with reparative properties. HUCB cells have been banked worldwide, and no harmful effects have been reported with their use as a cellular therapy. Unfractionated mononuclear HUCB cells are neuroprotective in experimental models of optic nerve trauma, brain, and spinal cord trauma, glaucoma, and cerebral hypoxic-ischemic injury. A single intraocular injection of unfractionated HUCB cells to rats 1 week following optic nerve crush (ONC) injury promoted RGC survival. None of the above studies address the impact of HUCB hematopoietic cells on axon regeneration, including optic nerve (ON) axon regeneration. Furthermore, the specific HUCB cell subsets responsible for neuroprotection, and their precise mechanism of action, remain to be definitively elucidated.

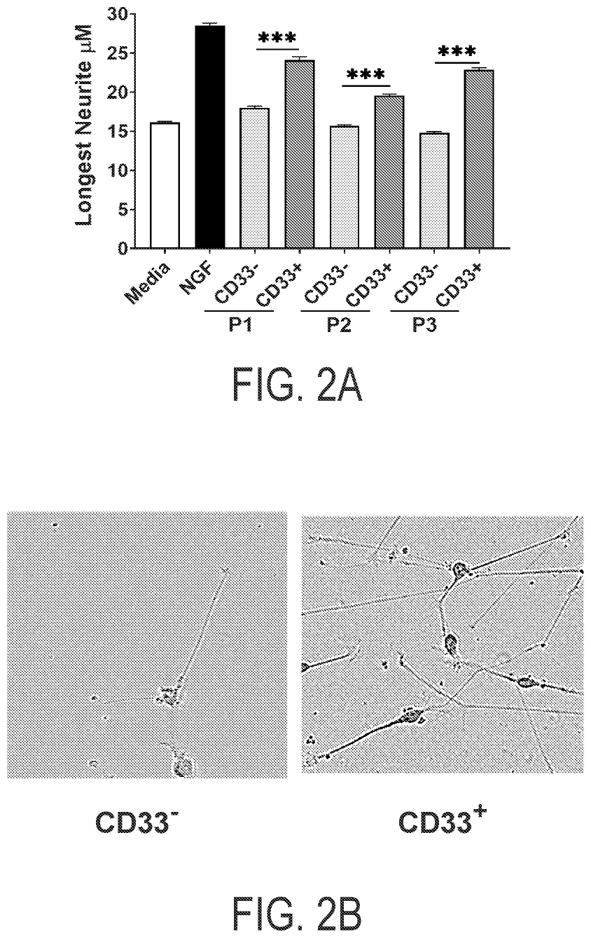

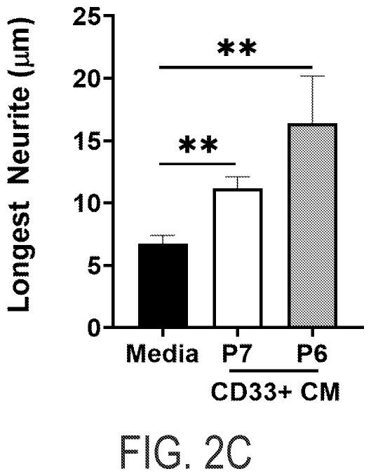

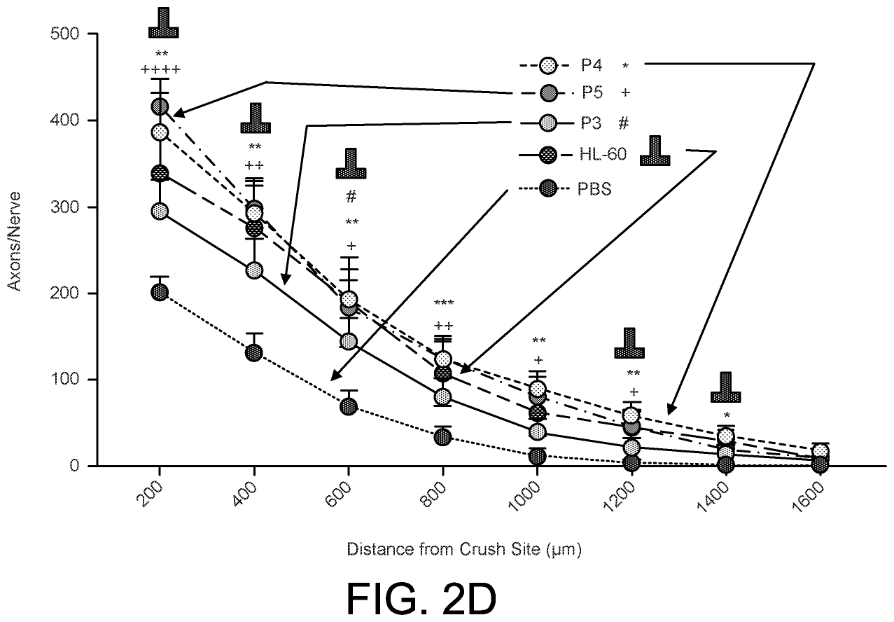

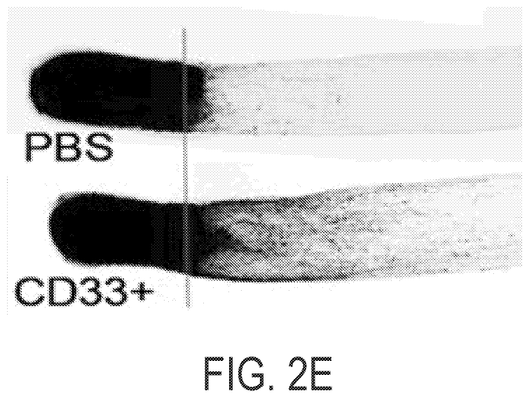

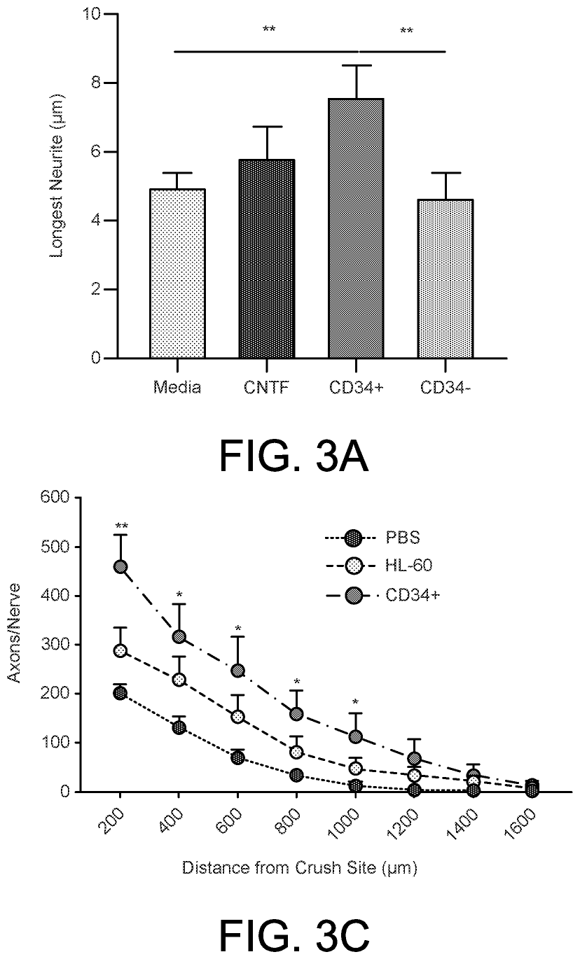

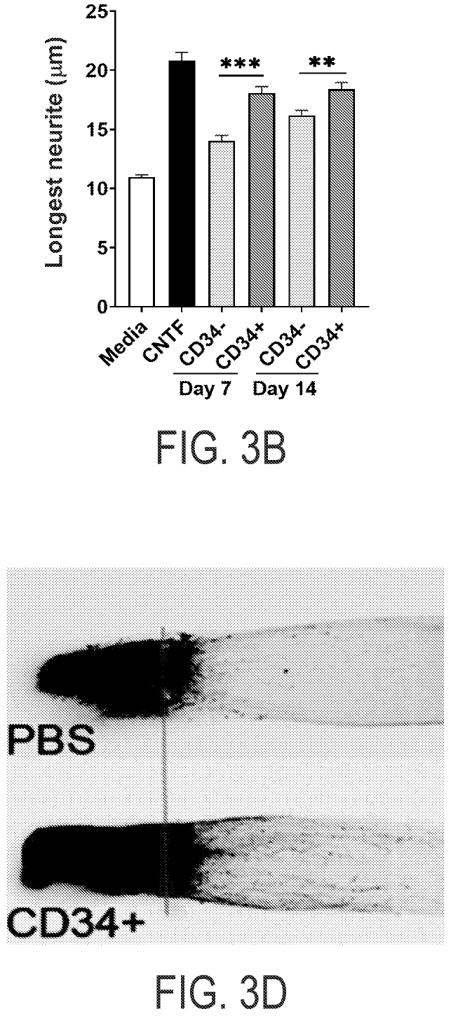

The study herein shows that primary CD33⁺ HUCB myeloid cells and short term CD33⁺CD34⁺ HUCB cell lines stimulate the outgrowth of adult RGC axons, in vivo as well as in vitro (FIGS. 2 and 3). Furthermore, in chimeric mice reconstituted with a human immune system, human CD33⁺CD34^{dim} myeloid cells accumulate in the posterior chamber of the eye in association with regenerating optic nerve axons. The current study interrogates the transcriptome, secretome and biological properties of reparative CD33⁺ HUCB cells and cell lines in detail. The mechanism of action underlying their neuroprotective/neuroregenerative effects is studied. The current study also explores strategies for enhancing the cytoprotective and pro-regenerative properties of primary HUCB CD33⁺ cells and short term HUCB CD33⁺CD34⁺ cell lines. Protocols and dosing regimens are evaluated for their use as a restorative cellular therapy for optic neuropathy and nervous system disorders, either alone or in combination with other complementary pro-regenerative interventions.

### Example 2. Interrogating the Transcriptome, Secretome and Biological Properties of Pro-Regenerative CD33⁺ HUCB Cells and Cell Lines

This study is designed to show that primary CD33⁺ HUCB myeloid cells and CD33⁺CD34⁺ HUCB myeloid cell lines express a cocktail of neuroregenerative/neuro-protective factors and immunomodulatory molecules. A combination of multi-color flow cytometry, transcriptomics, and proteomics are used to characterize neuroprotective/neuroregenerative HUCB myeloid cells in detail, gauge their diversity, and determine whether a subset within the broader pool CD33⁺ HUCB cells/cell lines is responsible for the therapeutic properties. The study determines whether CD33⁺CD34^{+ or -} HUCB cells and cell lines exercise their neuroprotective/neuroregenerative effects via the release of soluble factors or vesicles. The role of molecular mediators (identified via the above exploratory studies) is determined via loss of function experiments.

### Example 3. Therapeutic Potency of CD33+ HUCB Myeloid Cells for Management of Optic Neuropathy and CNS Disorders

This study tests if the efficacy of CD33⁺CD34^{+ or -} HUCB cells/lines as a cellular therapy for nervous system disorders and diseases can be enhanced by 3 general approaches. First, HUCB myeloid cells or lines are polarized and/or genetically engineered in a manner that expands their repertoire of cytoprotective and trophic factors, and that increases their longevity and retention at the sites of ocular injury. Second, the ocular microenvironment is modified via forced overexpression of selected factors that make it more receptive to the recruitment, polarization, and survival of reparative HUCB myeloid cells. Third, HUCB myeloid cell therapy is combined with complementary pro-regenerative interventions (ex. targeting of axon growth inhibitory pathways) to magnify therapeutic impact.

By identifying and characterizing neuroregenerative human myeloid cells, the experiments herein moves this line of research a step closer to the first clinical trials of novel immune cell therapies that are intended to block, or even reverse, neurological disability in human subjects secondary to conditions such as traumatic or ischemic-reperfusion optic nerve injury, traumatic brain or spinal cord injury, amyotropic lateral sclerosis, multiple sclerosis, Alzheimer's disease, Parkinson's disease, cerebral stroke, cerebral hemorrhage, optic neuritis, and glaucoma.

### Example 4. Neuroregenerative Functions

This study explores previously unknown neuroregenerative functions of human myeloid cells. The studies herein involve the investigation of singular subsets of immature myeloid cells, within the broader CD33⁺CD34⁺ or CD33⁺CD34⁻ HUCB myeloid cell population, that mediate neuronal protection and drive axon regrowth. At present, there are no therapies that reverse damage in the central nervous system. The strategy of deploying genetically engineered, in vitro polarized human myeloid cells as a cellular therapy to restore neurological function is innovative.

This study investigates multimodal approaches that combine unique innate immune cell therapies with complementary pro-regenerative interventions (ex. targeting of axon growth inhibitory pathways) to amplify overall therapeutic impact.

### Example 5. HUCB Myeloid Subsets that Promote Neuronal Survival and Axon Regeneration

Axonal transection and neuronal death are pathological features of neurological disorders caused by a wide range of insults, including physical trauma, glaucoma, neurodegeneration, autoimmune inflammation, and ischemic o hemorrhagic stroke. There is an unmet clinical need for therapies that enhance neuronal viability, trigger axon regrowth, and ultimately restore neurological functions. Although neuroinflammation is often depicted as detrimental, there is growing evidence that nonclassical, reparative leukocyte subsets and their products can be deployed to improve neurological outcomes. A well-established model of immune driven CNS axon regeneration involves crush injury to the murine optic nerve, resulting in extensive RGC loss and axonal transection, that is mitigated by intraocular (i.o.) injection of zymosan, a fungal cell wall extract. Similar to white matter tracts in the brain and spinal cord, transected axons in the optic nerve fail to undergo long distance regeneration. However, i.o. zymosan injection, at the time of ON crush injury, or up to 3 days afterwards, induces vitreal inflammation associated with rescue of RGC from death and regeneration of RGC axons. A novel subpopulation of immature Ly6G^{low} myeloid cells was shown to promote RGC survival and ON axon regrowth in that model, in part via secretion of a combination of nerve growth factor (NGF) and insulin-like growth factor-1 (IGF-1). These pro-regenerative murine cells express markers of alternative activation, including Arginase-1 (Arg1), IL-4 receptor a chain (IL-4Rα), and mannose receptor (CD206). Other populations of alternatively activated, nonclassical myeloid cells have been implicated in restorative pathways in animal models of cutaneous wounds, myocardial ischemia, and multiple sclerosis.

The discovery of reparative murine immune cells has led to the quest for a human immune-based cellular therapy that can mitigate damage in the human CNS, including the anterior visual system. Human umbilical cord blood (HUCB) cells have been interrogated as a source of immature immune cell subsets with healing properties. Unfractionated total or mononuclear HUCB cells (the latter of which contain immature T cells, B cells and myeloid cells) are neuroprotective in experimental models of optic nerve trauma, brain and spinal cord trauma, and cerebral hypoxic-ischemic injury. A single intraocular injection of unfractionated HUCB cells to rats 1 week following optic nerve crush (ONC) injury promoted RGC survival. Autologous, as well as allogeneic, unfractionated HUCB infusions have been tested as a neuroprotective cell therapy in early safety and feasibility clinical trials with human subjects diagnosed with autism spectrum disorder, cerebral palsy, stroke or spinal cord injury. A phase II trial of autologous cord blood cell infusion in children with cerebral palsy showed a potential beneficial effect on motor function and brain connectivity in the cohort that received the highest dose. At least 10 studies have been conducted using allogeneic HUCB to treat various CNS disorders across a range of HLA-matched/untyped units and cell doses. In the majority of those trials, immunosuppression was not administered post-infusion. Nonetheless, there were no reported serious adverse events definitively or probably related to allogeneic HUCB cell infusion, nor were there reports of delayed potential complications such as graft versus host disease.

None of the above studies address the impact of the HUCB cells on axon regeneration, including ON axon regeneration. Furthermore, the specific HUCB mononuclear cell subsets responsible for neuroprotection/repair, and their precise mechanism of action, remain to be definitively elucidated. Donor HUCB hematopoietic cells have a relatively short lifespan post-transfer compared with the duration of their therapeutic benefits, and do not transdifferentiate into CNS or retina resident cells. HUCB hematopoietic cells can secrete neuroprotective and trophic factors such as NGF and brain derived growth factor (BDGF), both of which have been detected in HUCB serum at high concentrations. Treatment with UCB serum-based eye drops preserves flash-electroretinogram responses and enhances the survival of retinal neurons in a rat model of retinal degeneration induced by high intensity light. The current study identifies specific HUCB myeloid subsets that promote neuron (including RGC) survival and axon regeneration.

### Example 6. CD33+ HUCB Cells

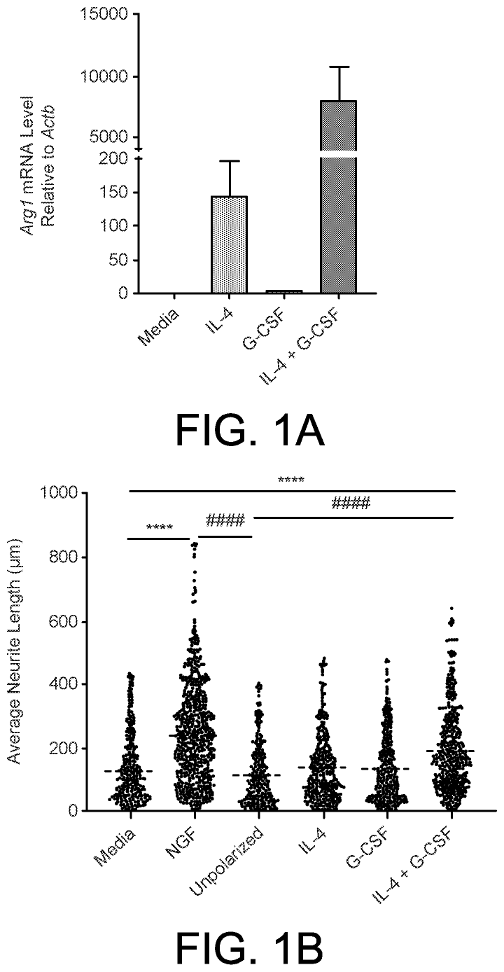

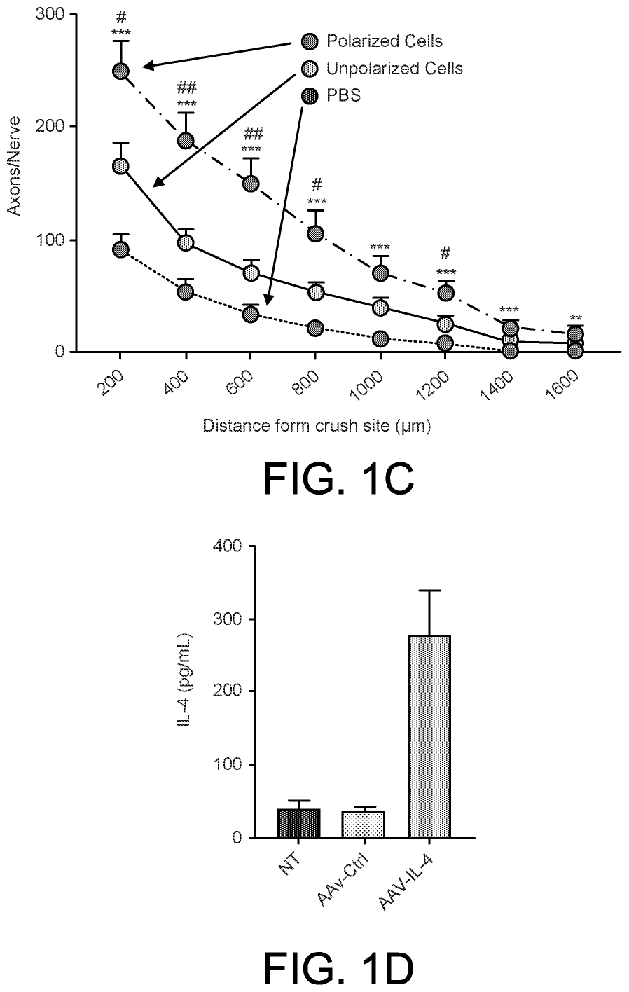

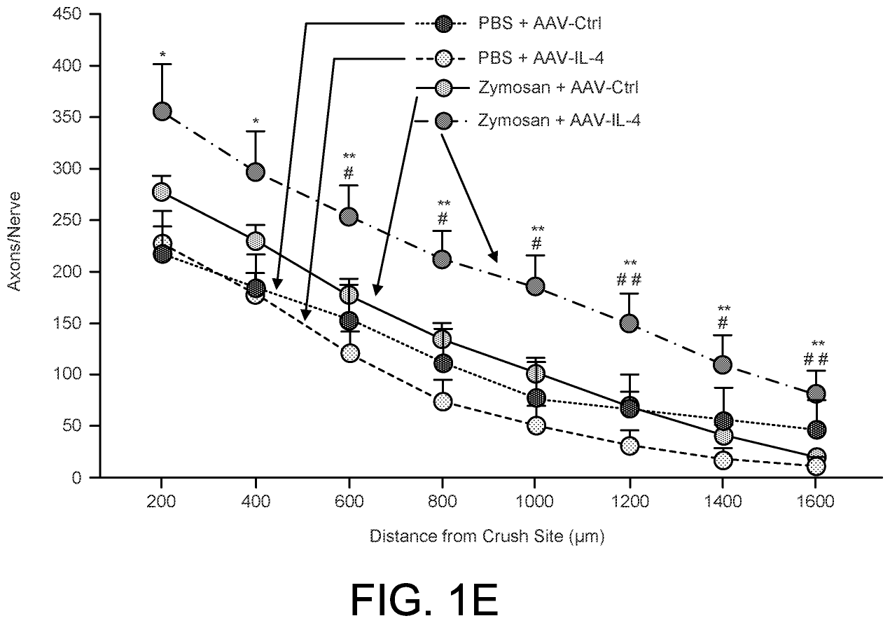

IL-4/G-CSF polarized Ly6G^{low} bone marrow cells promote RGC axon regrowth. Whether myeloid cells with RGC-protective/ON regenerative properties can be polarized from murine bone marrow (BM) progenitors ex vivo was tested. BM cells, flushed from the femurs of naïve C57BL/6 mice, were cultured with a series of different cytokine combinations, after which Ly6G^{low} cells were isolated and analyzed. Ly6G^{low} myeloid cells, polarized in the presence of recombinant IL-4 and G-CSF, display a nuclear morphology, cell surface phenotype and transcriptome reminiscent of the zymosan-induced pro-regenerative cells described in the previous publication (FIG. 1A, and data not shown). Importantly, IL-4/G-CSF polarized cells induce RGC axon regrowth in vitro and in vivo (FIGS. 1B and 1C). Next, IL-4 is over-expressed in the posterior chamber of the eyes of mice with ON crush injury, via i.o. injection of adeno-associated virus serotype 9 (AAV9)-derived vectors carrying an IL-4 transgene (FIG. 1D). This resulted in enhanced i.o. zymosan-induced ON axon regeneration (FIG. 1E), in association with an expanded number of alternatively activated Ly6G^{low} cells in the vitreous.

Primary CD33+ HUCB cells promote murine and human RGC axon regrowth. In order to assess the pro-regenerative properties of HUCB myeloid cell subpopulations, CD33⁺ or CD33⁻ cells were sorted from the HUCB mononuclear fraction of individual patients and co-cultured them with neonatal murine RGC (FIG. 2A), or RGC derived from human induced pluripotent stem cells (iPSCs) (FIG. 2B). Neurite outgrowth was measured 24 h and 48 h later, respectively. RGC were cultured with nerve growth factor (NGF) as a positive control. CD33⁺, but not CD33⁻, HUCB cells from each patient independently promoted neurite outgrowth by both murine and human RGC compared with the media only control (FIGS. 2A and 2B). Conditioned media (CM) from cultured CD33⁺ HUCB cells also triggered neurite outgrowth of human RGC (FIG. 2C). Next, the efficacy of CD33⁺ HUCB cell therapy was investigated in vivo. CD33⁺ HUCB cells, purified from individual donors, were injected separately into the vitreous of different groups of RAG1^{-/-} mice, on the day of ON crush injury and 48 h later. ONs were harvested 2 weeks following crush injury and the density of regenerating axons was measured at serial distances from the crush site. Consistent with the results of the neurite outgrowth assays, CD33 HUCB cells from 3 out of 3 patients triggered RGC regeneration of the crushed ON upon adoptive transfer (FIGS. 2D and 2E).

Short term CD33+CD34+ HUCB cell lines exhibit reparative properties in the eye. Analogous experiments to those in FIG. 2 were performed using short term CD33⁺CD34⁺ HUCB cell lines. CD34⁺ cells were isolated from the CD33⁺ HUCB mononuclear cells of individual donors using the EasySep Human CD34 Positive Selection Kit II (STEMCELL technologies). Cell lines were expanded, in parallel, with recombinant myeloid growth factors (described under Experimental Approach) for 7-14 days, and then harvested for use in neurite outgrowth assays or adoptive transfer experiments. CD34⁺, but not CD34⁻, short term cell lines stimulated significant neurite outgrowth of explanted neonatal mouse RGC (FIG. 3A) as well as human iPSC-derived RGC (FIGS. 3B, 3C), and drove ON axon regeneration in vivo following adoptive transfer (FIGS. 3D, 3E).

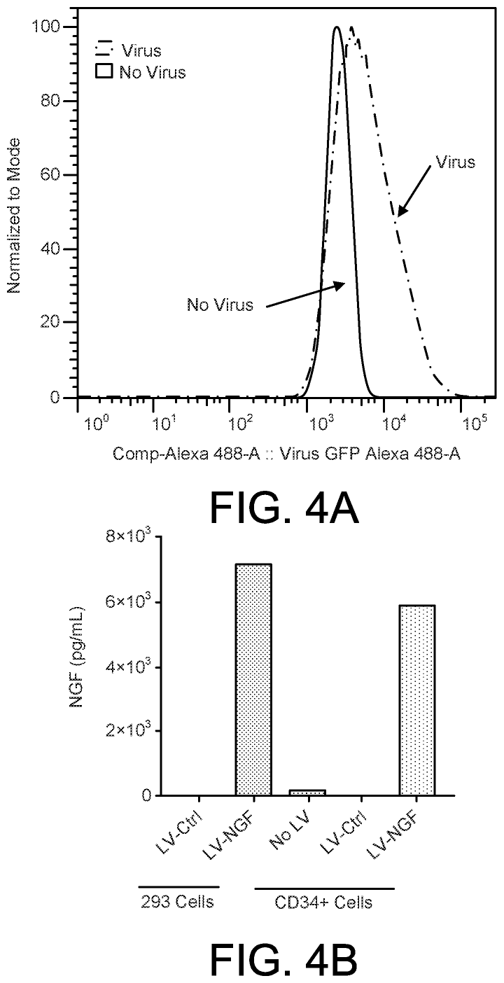

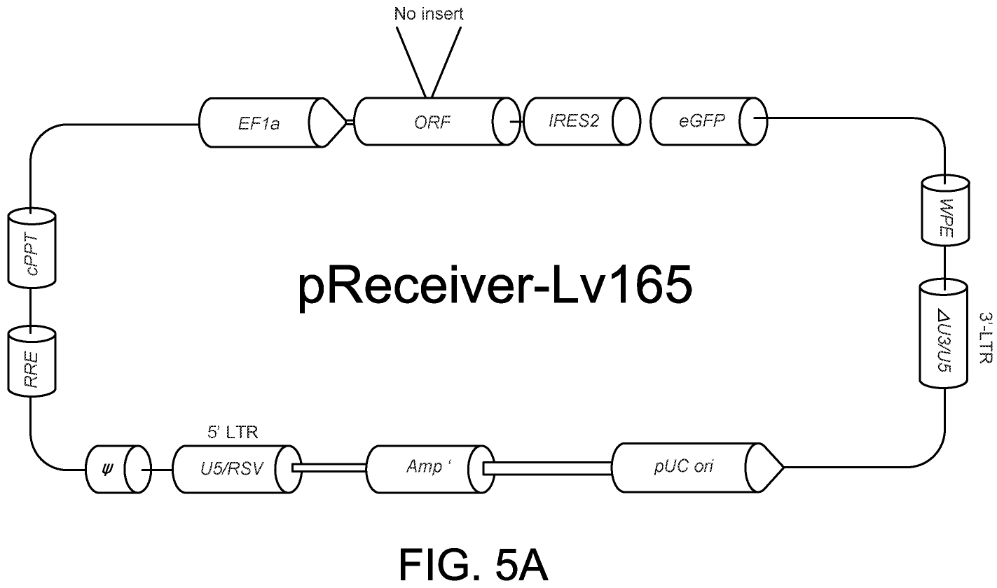

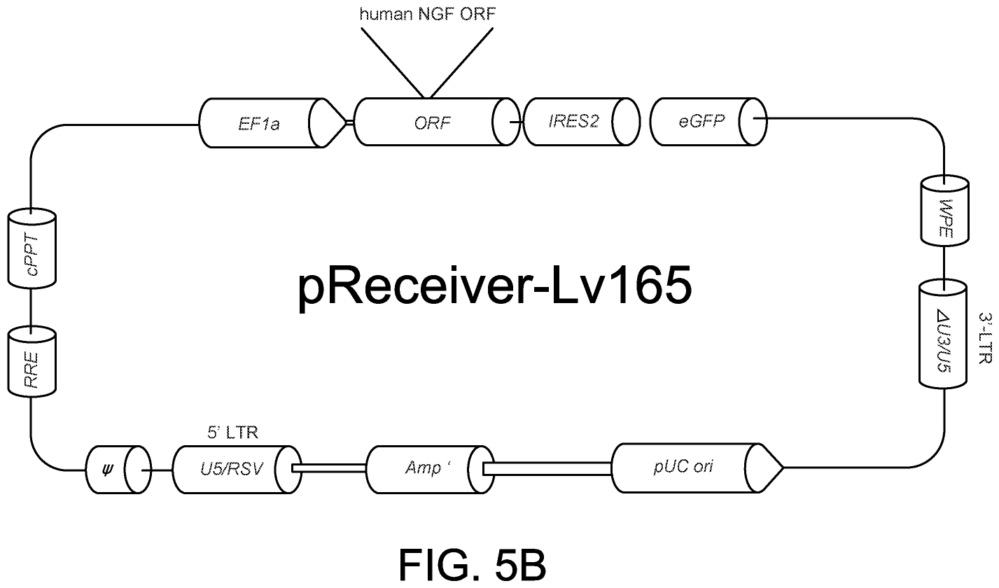

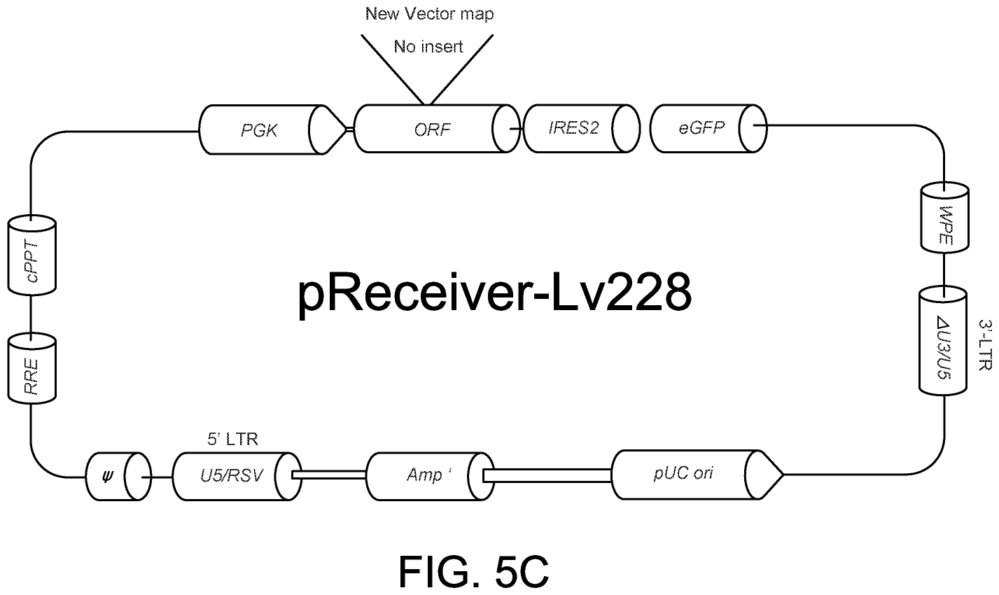

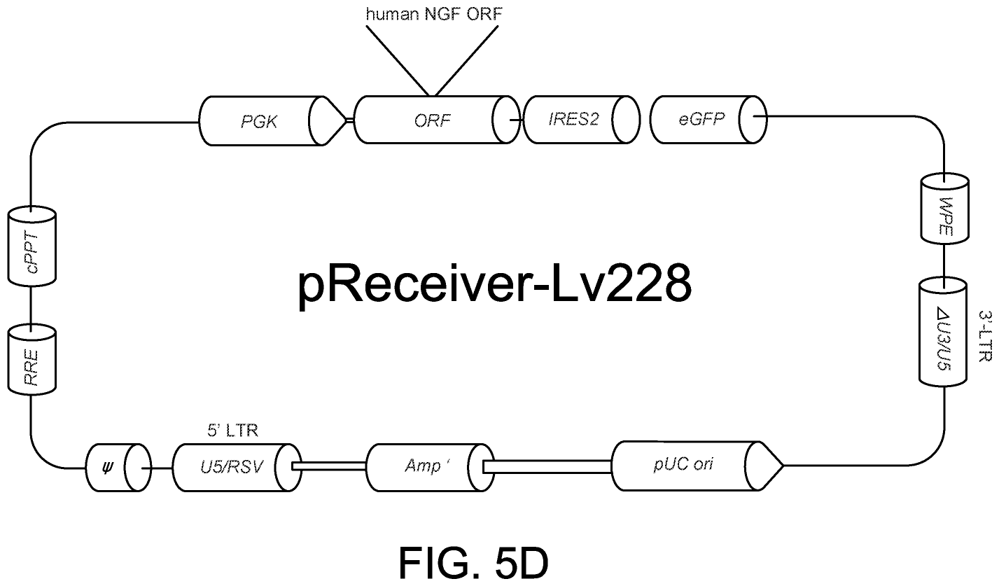

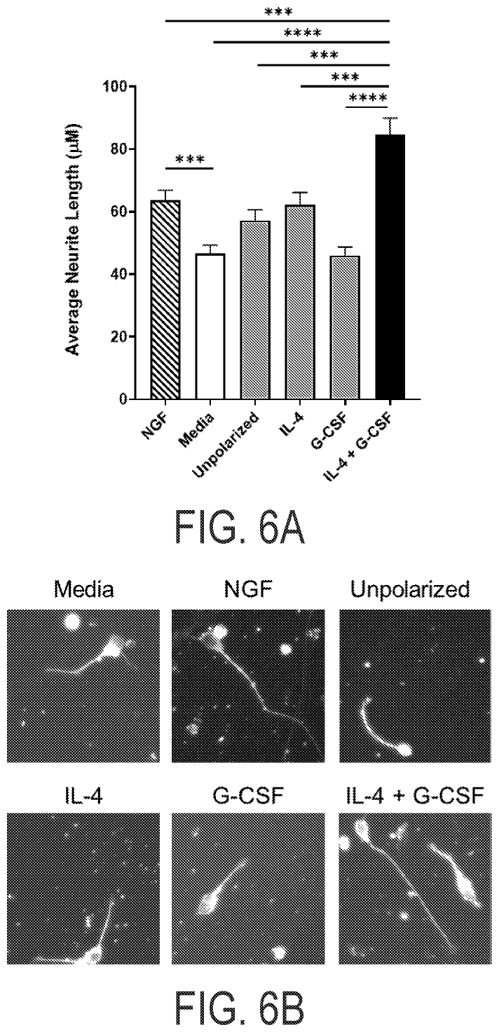

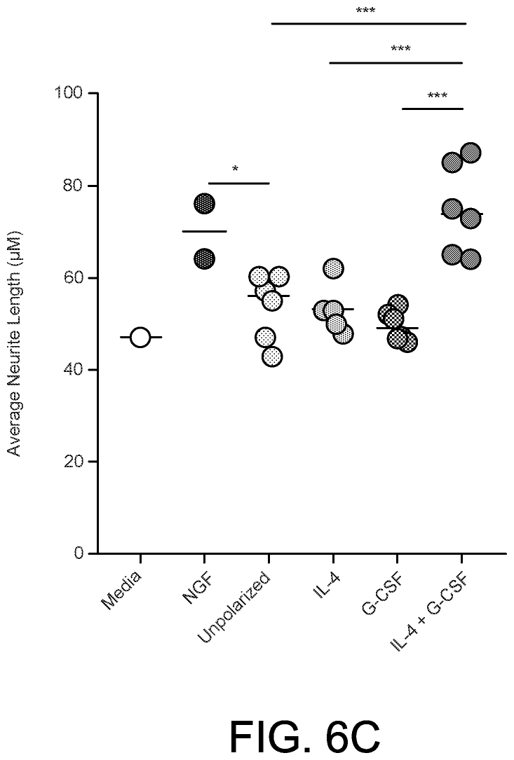

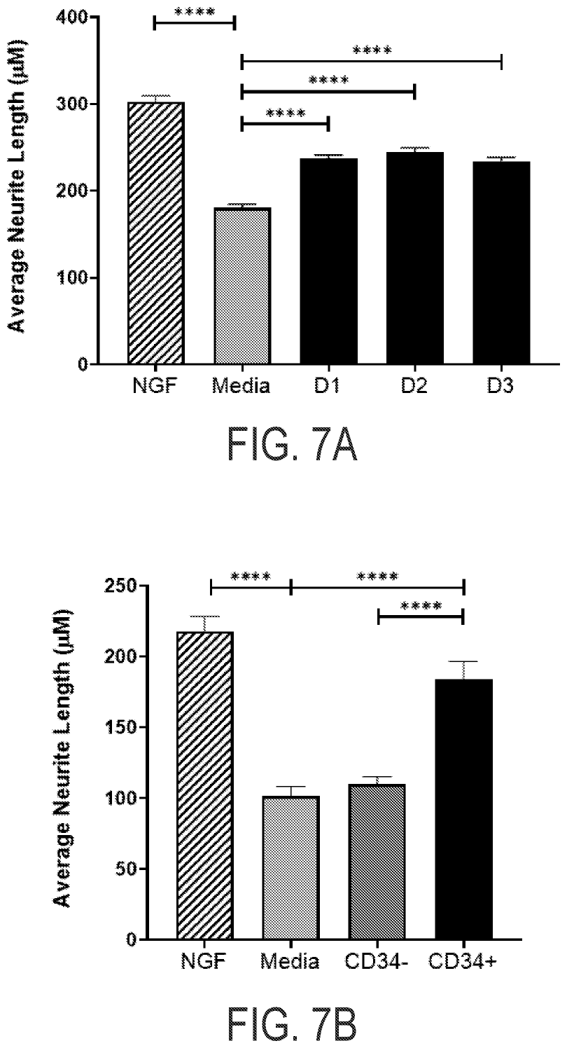

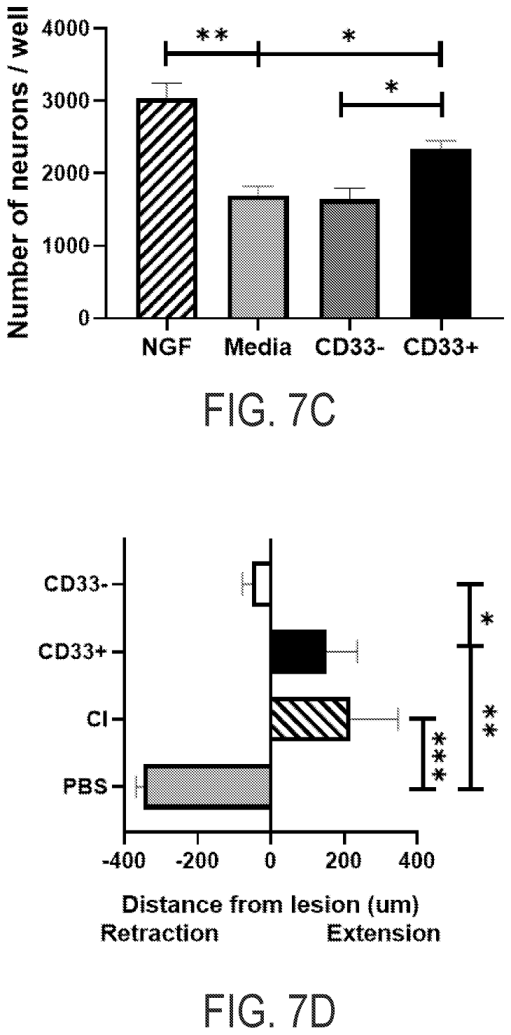

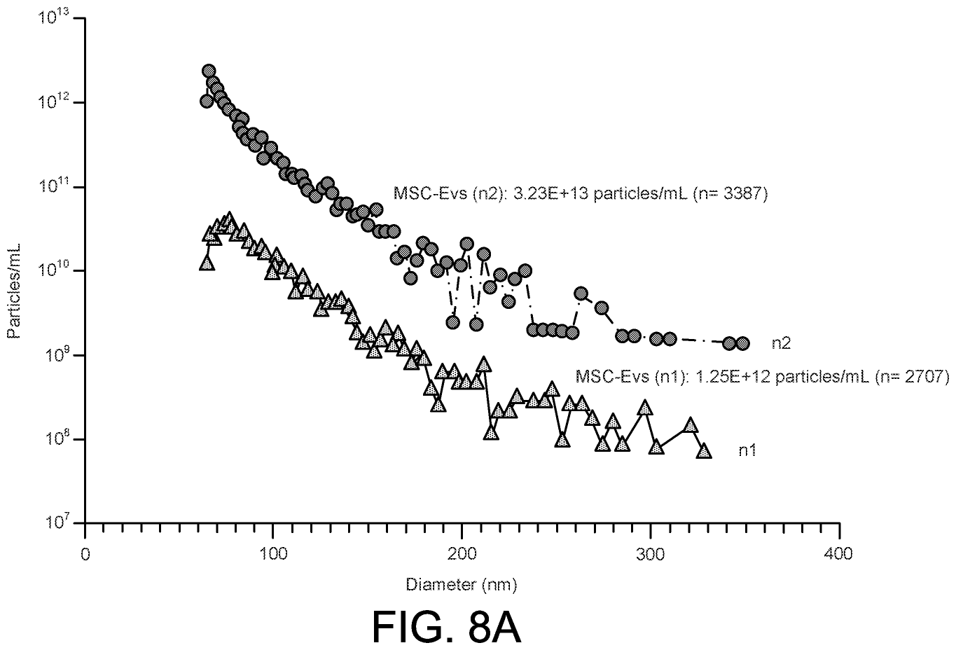

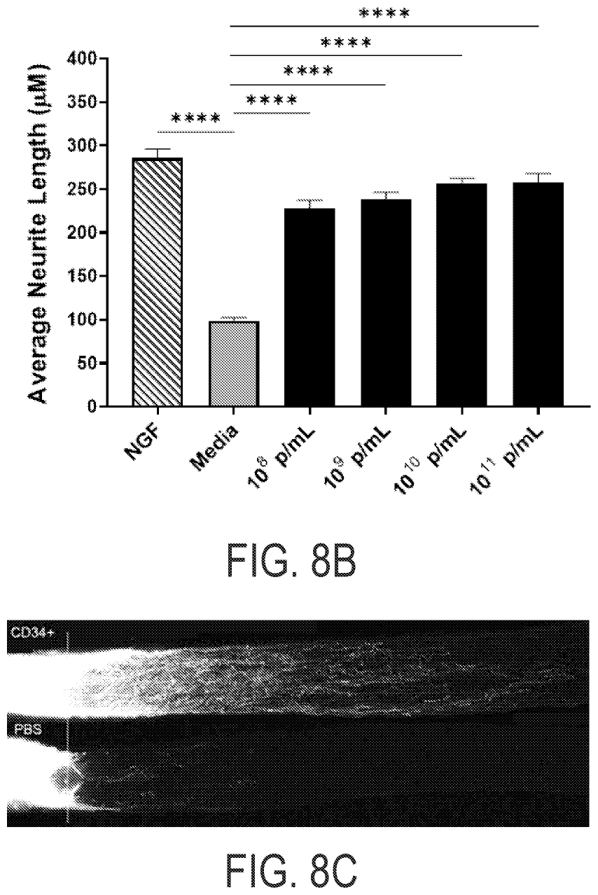

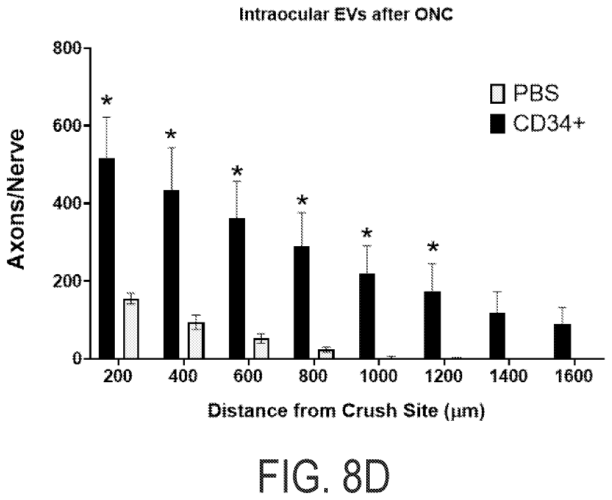

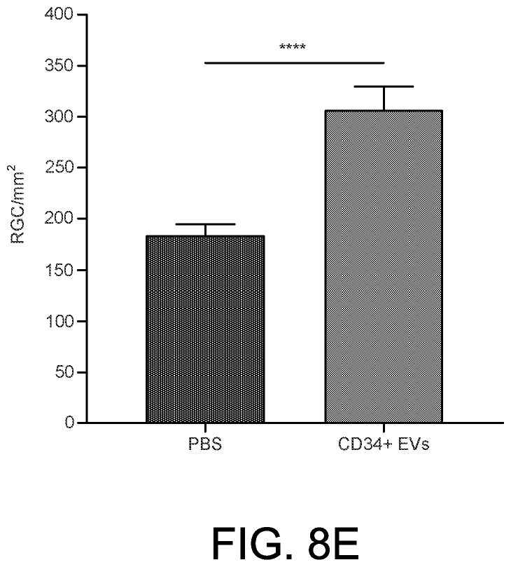

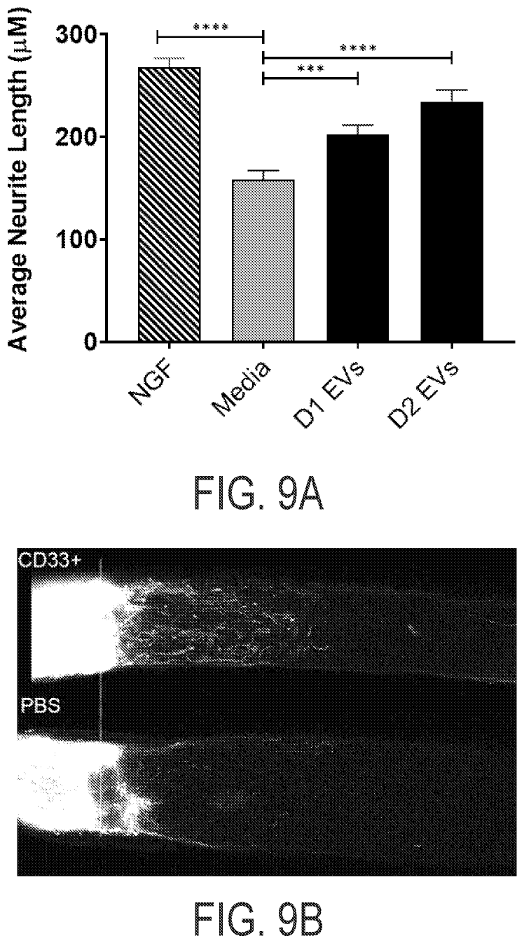

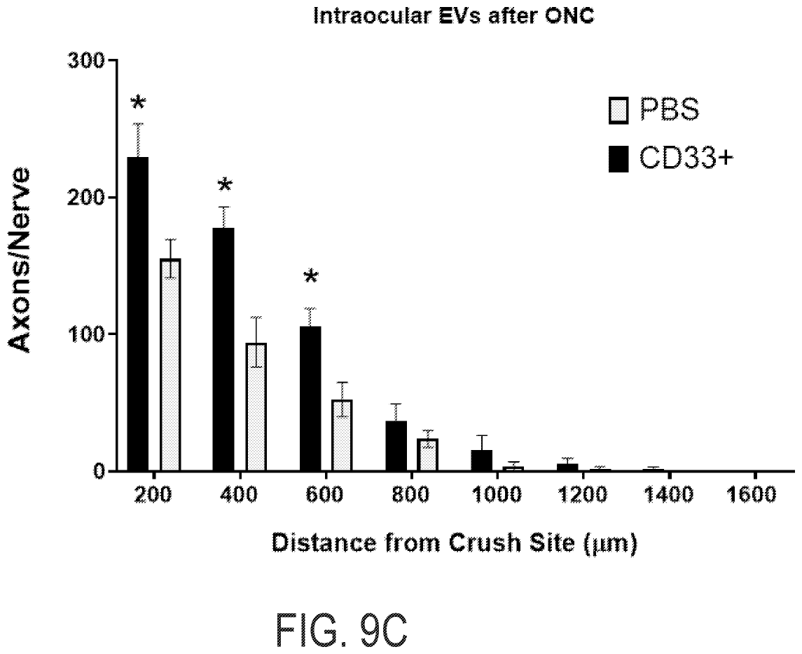

CD33⁺ HUCB can be genetically engineered to express recombinant growth factors and other molecules. Also provided herein is a method to genetically engineer primary CD33′CD34⁺ or CD33⁺CD34⁺ HUCB donor cells/cell lines in a manner that expands their repertoire of cytoprotective and growth factors. To investigate the feasibility of that approach, freshly isolated CD33⁺CD34⁺ HUCB cells were transduced with lentivirus (LV) vectors either carrying the NGF gene in tandem with a GFP reporter or the GFP reporter gene only (2×10⁶ TU/mL; MOI 50). Fresh media was added to all wells at 24 h. After another 48h, cells and supernatants were harvested and analyzed by flow cytometry and NGFβ ELISA, respectively. It was found that 40-45% of transduced HUCB cells expressed the GFP reporter (FIG. 4A). Supernatants collected from cultured HUCB cells that were transduced with the NGF-GFP LV vectors, but not GFP only vectors, contained large quantities of NGFβ protein (FIG. 4B).

### Example 7. Investigating the Transcriptome, Secretome and Biological Properties of Pro-Regenerative CD33⁺CD34⁺ or CD33⁺CD34⁻ HUCB Cells

CD33⁺ HUCB myeloid cells may produce a cocktail of cytoprotective and growth factors and immunomodulatory molecules that mediate neuroprotection and drive axon regeneration.

Unfractionated HUCB cells are neuroprotective in multiple animal models of CNS trauma and disease, including optic nerve crush injury in rats. There have been over 20 early phase clinical trials of HUCB infusion in human subjects with neurological disorders, all with a favorable safety profile. Some of these studies have reported promising signals in terms of clinical outcomes. Clinical trials of HUCB cells have yet to be conducted in ocular conditions. This study found that primary CD33⁺ myeloid cells, and short term CD33⁺CD34⁺ cell lines, derived from the mononuclear fraction of HUCB, promote RGC survival and ON axon outgrowth upon i.o. injection into immunodeficient mice (FIGS. 2 and 3). Next, a granular analysis is performed on pro-regenerative CD33⁺ HUCB primary cells and CD33⁺CD34⁺ cell lines to elucidate their mechanism of action (MOA). This analysis can identify the specific HUCB myeloid subsets responsible for the reparative properties that are observed in FIGS. 2 and 3. An in-depth characterization of pro-regenerative HUCB myeloid cells is a necessary early step in the development of protocols for their preferential expansion, enrichment, and isolation as a cellular therapy. A deeper understanding of the MOA of the HUCB myeloid cells can also lead to the development of alternative pharmaceutical or gene-based interventions that enhance RGC viability and reverse optic neuropathy. For example, interrogation of the secretome of pro-regenerative HUCB cells could lead to the discovery of a particular combination of growth factors that act synergistically to promote repair within the CNS and/or eye. Such a cocktail can conceivably be delivered using nanoparticles or genetic vectors.

HUCB myeloid cells and cell lines, isolated or derived from individual donors, are employed and analyzed separately, and used as biological replicates. Every experiment is performed with at least 3 biological replicates. In addition, experiments with each biological replicate are repeated at least 3 times (technical replicates) to ensure reproducibility. All assays are performed with de-identified samples; investigators who measure outcomes are blinded to the experimental groups. Mice are selected to ensure equal numbers of males and females, where feasible. Of note, in adoptive transfer studies in mice, no differences were found when experiments were performed with males or females, including inflammatory responses and extent of RGC survival/axonal regeneration. All experiments utilizing neutralizing antibodies, shRNA, or small molecule antagonists include negative controls with isotype matched control antibodies, scrambled shRNA and inert small molecules, respectively. For adoptive transfer studies, 16 nerves and retinae from n=8 mice per group provide more than 80% power to detect a 2-fold change in the density of regenerating axons or frequency of viable RGC between groups with CV=50% at α=0.05/10=0.005, adjusting for multiplicities. Estimates are based on data to infer coefficients of variation (CV) or standardized effect sizes based on prior experience and designed to achieve ≥80% power.

HUCB cells are acquired from the Leukemia Tissue Bank Shared Resource (LTBSR) at OSU. The LTBSR regularly collects and de-identifies HUCB cells from the full-term placenta of healthy women who are admitted to the Labor and Delivery ward of the Ohio State Maternity Center. Informed consent is given by donor mothers prior to collection. Primary CD33⁺ HUCB cells (FIG. 2) and CD33⁺CD34⁺ HUCB cell lines (FIG. 3) are used, and their efficacy and translatability as a cell therapy for CNS disorders and injuries are compared. In the experiments shown herein, the mononuclear fraction of whole HUCB cells is enriched by Ficoll density gradient centrifugation. Primary CD33⁺ cells are isolated by FACS sorting or MACS magnetic bead purification and used in the assays and adoptive transfer experiments described below. Of note, these cells retain regenerative properties when thawed after storage at −80 C. CD33⁻ subsets are isolated in parallel for use as controls. For some experiments, CD33⁺CD34⁺ cells are purified from the CD33 HUCB mononuclear fraction using the EasySep Human CD34 Positive Selection Kit II (STEMCELL technologies), and expand them in serum free medium (containing bovine serum albumin, insulin, transferrin and other supplements in Iscove's MDM; StemSpan SFEMII, STEMCELL Technologies) in the presence of recombinant human stem cell factor, thrombopoietin, G-CSF, IL-4, TGF-β, and/or GM-CSF (StemSpan Myeloid Expansion Supplement, STEMCELL technologies). After 14 days the cells are harvested and characterized in terms of cell surface phenotype, transcriptome, secretome, and biological functions, or used as donor cells in adoptive transfer experiments, as with the primary CD33⁺ HUCB.

Characterization of CD33⁺ myeloid cells. Primary CD33⁺ HUCB cells and CD33⁺CD34⁺ HUCB short term cell lines, as well as FACS sorted subsets of those cells/lines, are characterized via a series of assays, detailed below. For negative controls, CD33⁻ HUCB cells or mature CD33⁺ PBMC from healthy adults are used, which lack neuroregenerative properties (FIGS. 2A and 3A). For a positive control, the human HL60 myeloid cell line is used (derived from a patient with acute myelogenous leukemia), which has been demonstrated to promote RGC axon regrowth in vitro and in vivo.

Flow cytometry. A four laser, 21 color flow cytometer is used, which allows extensive characterization of the cell surface phenotype and intracellular contents of pro-regenerative primary CD33⁺ HUCB cells and CD33⁺CD34⁺ cell lines. In addition to standard lineage markers (CD33, CD11b, CD14, CD15, CD16, CD3, CD4, CD19, CD56, CD11c), the flow antibody panels collectively encompass markers of maturation/activation (CD34, CD38, CD117, c-kit, CD45RA, CD123, CD10, CD66b, HLA-DR), alternative activation (CD206, IL-4 receptor a chain, CD163, and intracellular Arg-1), chemotactic receptors (CCR2, CCR3, CCR4, CCR5, CXCR1/2, CXCR3, CXCR4, CXCR6). In some instances, intracellular cytokines (IL-4, IL-10) or growth factors (NGF, BDNF, CNTF) are stained. In many of the panels, monoclonal antibodies specific for non-myeloid lineage markers are conjugated to the same fluorochrome for dump gating, in order to maximize the number of colors that can be used to detect other markers. Flow cytometry panels are designed with BD resources such as Spectrum Viewer, absorption and emission spectra, and the buffer compatibility tool.

Transcriptomics. Single cell RNA sequencing (scRNAseq) is performed for analysis of primary CD33⁺ HUCB cells and short term CD33⁺CD34⁺ HUCB cell lines. If flow cytometric analyses reveal phenotypically diversity among these HUCB populations (based on differential expression of markers of alternative activation, maturation, and lineage commitment), scRNAseq can be performed on selected FACS sorted subpopulations to increase the resolution of these studies. The approach described in previous work is used. Briefly, cDNA libraries are constructed using 10× Chromium™ Next GEM Single cell 5′ Reagents v2, quantified using the Illumina Library Quantification Kit, and sequenced on the Illumina NovaSeq 6000 Sequencing System. For single-cell expression profiling, reads are aligned to the reference genome using CellRanger counts software. Low-quality cells are excluded in an initial quality control (QC) step by removing genes expressed in fewer than three cells. Cells that express<200 genes, >2500 genes, or >0.10% of mitochondrial-associated genes are also removed. The data are normalized using log normalization and scale factor 10,000 using the Normalize Data function in Seurat. To identify the differentially expressed features, the FindMarkers function is used with min.pct argument set to 0.25, which filters out genes expressed in <25% of the cells. Multidimensional reduction, batch correction, cell clustering and gene signature discovery are performed using Seurat with default settings. Gene signatures are assigned to selected cell clusters using the unbiased “Gene Modules” method.

Expression levels of genes of interest are confirmed via digital RT-PCR. UMAP clusters are matched, defined via transcriptomic profiling, with HUCB subpopulations delineated by flow cytometric analysis of cell surface marker expression. Selected HUCB subsets that correspond to UMAP clusters are FACS sorted for functional assays and adoptive transfer experiments.

Secretomics. Conditioned media is harvested from cultured primary CD33⁺ HUCB cells and short term CD33⁺CD34⁺ cell lines, as well as selected FACS sorted subsets of those populations. A panel of growth and immunomodulatory factors are measured using magnetic bead-based multiplex assays on the Luminex platform, or sandwich ELISAs when necessary. Among the analytes, nerve growth factor beta (NGFβ), ciliary neurotrophic factor (CNTF), brain-derived growth factor (BDNF), and glial derived neurotrophic factor (GDNF) can be measured, using the Human Neurotrophic Factors 4-Plex Human Panel (ThermoFisher). Platelet derived growth factor (PDGF), Fibroblast growth factors (FGF), TGF-β, and IL-10 can be measured using customized panel or ELISA. Additional analytes are selected based on the transcriptomic experiments mentioned above.

Nuclear morphology and cellular granularity. Cytospins of FACS sorted subsets of CD33⁺ HUCB cell and short term CD33⁺CD34⁺ cell lines are prepared for demonstrating cytoprotective and pro-regenerative characteristics (informed by the results of flow cytometric and single cell transcriptomics described above, and functional assays described below). The slides are stained with Wright-Geimsa solution and imaged under an Olympus IX71 inverted microscope. Primary CD33⁺ HUCB cells and short term CD33⁺CD34⁺ HUCB cell lines contain 1 or more subpopulations that exhibit features indicative of a relatively immature stage of differentiation and alternative activation, in association with growth, neuroprotective and/or immunomodulatory molecules.

### RGC Protective/ON Regenerative HUCB Subsets.

Assessment of the cytoprotective and pro-regenerative properties of HUCB subpopulations in vitro. Neurite outgrowth assays are performed with FACS sorted subpopulations of primary CD33⁺ HUCB cells and short term CD33⁺CD34⁺ HUCB cell lines, prioritizing subpopulations that are enriched in alternative activation markers and/or trophic and cytoprotective factors (as revealed by the flow cytometric studies and scRNAseq analysis described above). The neurons to be used in neurite outgrowth assays are neonatal mouse RGC or human iPSC derived RGCs, both of which were found to be responsive to pro-regenerative factors secreted by unfractionated CD33⁺ HUCBs (FIGS. 2 and 3). Assays with murine RGC are critical to gauge the likelihood that HUCB subsets can drive ON axon regeneration upon transfer into immunodeficient mice (see next section). Neurite outgrowth assays are performed with the human iPSC-derived RGC in parallel, in order to more accurately gauge the ultimate translatability of HUCB cell therapy to the clinic. For positive controls, RGC is cultured with the HL60 cell line, unfractionated CD33⁺ HUCB cells, or recombinant CNTF (FIGS. 2 and 3). For negative controls, RGC is cultured with media alone, CD33⁻ HUCB cells or CD33⁺ PBMC. Neurite outgrowth assays are performed with or without a transwell separating the HUCB cells from the RGCs. If the transwell experiments indicate that a particular HUCB subpopulation secretes pro-regenerative soluble factors, the outgrowth assays are repeated with conditioned media (CM) harvested from that subpopulation. For negative controls, unconditioned media, or CM harvested from cultured CD33⁻ HUCB cells are used. In complementary studies, RGC viability is measured by culturing retinal explants or iPSC derived RGCs with control or conditioned media for 1-7 days. Whole mounts or cultured cells are immune-stained with monoclonal antibodies against βIII Tubulin or RBPMS, and RGC counted with ImageJ software (NIH). In order to determine if pro-regenerative/RGC-protective factors are proteins, the HUCB cell CM is heat shocked and tested alongside with unshocked CM. CM that contain pro-regenerative proteins are subjected to secretomics assays, as described above.

In order to definitively identify those soluble factors that contribute to the MOA of HUCB myeloid cells, and/or sorted subpopulations, neurite outgrowth and RGC viability assays are repeated with CM in presence of neutralizing antibodies, shRNA or small molecule antagonists that target cytoprotective molecules or growth factors. The selection of the factors to target is informed by the results of secretomic assays, outlined above. In parallel, neurite outgrowth assays are conducted with CM in the presence of isotype matched control antibodies, scrambled shRNA, or inert small molecules, as negative controls.

Assessment of the cytoprotective and pro-regenerative properties of HUCB subpopulations in vivo. CD33⁺ or CD33⁺CD34⁺ HUCB subsets, shown to be neuroprotective/pro-regenerative in neurite outgrowth assays, are FACS sorted and adoptively transferred into the eyes of immunodeficient (RAG1 knock-out) mice with ON crush injury. Cells are injected using a Hamilton syringe with a 30 G needle. Control mice are injected i.o. with vehicle alone, CD33⁻ HUCB cells or CD33⁺ PBMC. Four to six weeks later retina and optic nerves are harvested to measure the frequencies of viable RGCs and regenerating ON axons. Retinal whole mounts are stained with goat anti-mouse brn3a (santa-cruz), followed by Alexa Fluor 488-conjugated donkey anti-goat secondary antibody (Invitrogen). Longitudinal sections (10 um thick) of paraformaldehyde fixed, OCT-embedded optic nerves re stained with polyclonal rabbit anti-GAP43 (abcam), followed by an Alexa Fluor 488-conjugated donkey anti-rabbit secondary antibody (Invitrogen). GAP43 is upregulated in regenerating axons. Retina and optic nerve sections are imaged using an Olympus IX83 inverted confocal microscope. Brn3a⁺ RGCs are counted using Image J software in 8 fields per case distributed in 4 quadrants of the eye at prespecified distances from the optic disc. Regenerating GAP43⁺ axons are counted at each 0.2-mm interval past the injury site, up to 1.6 mm, using a superimposed grid (3-5 sections/nerve). The number of labeled axons per section is normalized to the width of the section and converted to the total number of regenerating axons per optic nerve.

For a physiological readout of visual function, the electrical activity of the retina is assessed using pattern electroretinography (PERG). For every experimental group, PERG (Diagnosys Celeris System) is performed at baseline (pre-injury) and then weekly, in an electrically shielded darkroom, with dark adapted, anesthetized mice on a thermostatically controlled built-in warming plate. A black and white reversing checkerboard pattern with a check size of 1° is displayed on light guide electrode-stimulators placed directly on the ocular surface of both eyes and centered with the pupil. The visual stimuli are presented at 85% contrast and mean luminance of 500cd/m², with a spatial frequency 0.059 cycles/deg and a temporal frequency 1 Hz. Two consecutive recordings of 400 traces are averaged to achieve one readout. Waveforms are analyzed with MATLAB software (Mathworks) to identify the major P1 and N2 waves, and to calculate peak-to-trough P1-N2 amplitudes.

In initial experiments, HUCB cells are administered according to the dosing regimen described in the previous work and the data herein (FIGS. 2 and 3) (i.e., 3×10⁵ cells in 3 ul, i.o., on days 0 and 3 post-ON crush injury). If administration of a particular HUCB subset according to that regimen is therapeutically effective, the experiment is repeated with a single dose, and/or systematically reduce the cell concentration per dose. Subsequent experiments are conducted using the lowest therapeutic dose of HUCB cells. In addition, in order more accurately simulate real life clinical scenarios, HUCB cell adoptive transfer is delayed until after crush injury. The time interval between the injury and adoptive transfer s increased systematically. Of note, it was found that the i.o. transfer of zymosan-induced murine Ly6G^{low} cells can be delayed by up to 12 hours post ON crush injury and still produce a significant therapeutic effect. In some experiments, vitreal fluid is obtained from mice at serial time points following adoptive transfer of HUCB cells, and a panel of growth and cytoprotective factors is measured via Luminex and ELISA.

Once therapeutically beneficial HUCB subsets are identified, neutralizing antibodies, shRNAs or small molecule antagonists against growth/cytoprotective factors are administered to mice with ON crush injury, by i.o. or i.v. injection, beginning on the day of HUCB subset transfer. The targeted factors are selected based on the results of the scRNAseq, secretome and neurite outgrowth assays described above. Control groups are treated with the appropriate isotype matched control antibodies, scrambled shRNA or inert small molecules. After 4 weeks, PERGs are repeated and retina and ONs harvested to enumerate the frequency of RGC and density of regenerating axons, using protocols and methodologies described above.

Extracellular vesicles. Extracellular vesicles (EVs) released by HUCB have been shown to mediate anti-impairment and pro-regenerative effects, such as amelioration of bone loss in osteoporotic mice and acceleration of wound healing. EVs derived from CD34⁺ hematopoietic HUCB cells contain a distinctive miRNA profile, including miR-221. miR-221 stimulates proliferation of rat neural stem cells and accelerates diabetic wound repair. The current study tests whether HUCB myeloid cells promote repair in the eye via secretion of EVs that carry cargo containing non-coding RNAs and/or growth factors with cytoprotective/pro-regenerative properties. To assess this effects, neuro-reparative CD33⁺CD34^{+ or -} HUCB cell/line subsets (identified using neurite outgrowth assays and adoptive transfer experiments) and CD33⁻ counterparts (used as negative controls) are cultured in serum free media. The CM is harvested at serial time points (24, 48 and 72 h) and concentrated using an Amicon ultra-15 centrifugal filter. EVs are isolated using size exclusion columns (SEC, qEVs) according to the Minimal Information for Studies of Extracellular Vesicles 2018 (MISEV2018) guidelines. Purified fractions 7-12 are collected and characterized via western blot, probing for exosome-enriched proteins (e.g. CD9, CD63, CD81) and selected growth and cytoprotective factors (e.g. BDNF, IGF-1). RNA is extracted from EV and selected miRNAs (e.g., miR-221) measured by qRT-PCR. Transmission electron microscopy (TEM) is used to demonstrate the lipid bilayer of the EVs. Nanoparticle concentration and size are determined by microfluidic resistive pulse sensing (MRPS). Fractions with the highest EV concentrations are pooled to be used in neurite outgrowth assays. iPSC derived human neurons are cultured in the presence of the EVs from each group, or in media alone, for 24 hours. Neurite outgrowth and RGC survival are then measured, as detailed above.

This study is designed to identify one or more subpopulations of myeloid cells among primary CD33⁺ HUCB cells and CD33⁺CD34⁺ HUCB cell lines, that enhance RGC survival, induce RGC axon regrowth and improve RGC function (i.e., increase PERG amplitudes). Pro-regenerative HUCB cells express markers indicative of an immature stage of differentiation and alternative activation. These experiments elucidate the soluble factors/vesicles that underlie their therapeutic effects.

### Example 8. Efficacy of CD33⁺ HUCB Cell Therapy for Management of Nervous System Disorders and Injury

CD33⁺ HUCB cell infusions that are effective in repairing the damaged CNS can be augmented by: (i) polarizing or genetically engineering primary CD33⁺ HUCB donor cells or CD33⁺CD34⁺ HUCB donor cell lines in a manner that expands their repertoire of cytoprotective and growth factors, thereby enhancing their reparative properties, or that heightens their expression of anti-apoptotic molecules and chemotactic receptors, thereby prolonging their longevity and retention in the eye; (ii) modifying the CNS or ocular microenvironment by over-expressing polarizing factors and chemoattractants that stabilize the phenotype of reparative HUCB cells and accelerate their accumulation locally, and/or (iii) combining HUCB cell infusion with independent pro-regenerative interventions (ex. blockade of intrinsic axon growth inhibitory pathways), thereby magnifying therapeutic impact via synergistic mechanisms.

Non-classical or alternatively activated murine myeloid cells have been generated in vitro by stimulation of bone marrow cells with recombinant cytokines, such as IL-4, IL-10 and TGF-β. In a number of experimental paradigms, myeloid cells polarized with one or more of those cytokines are reparative. Previous work has found that polarization of murine bone marrow cells with a combination of IL-4 and G-CSF transforms them into pro-regenerative Ly6G^{low} myeloid cells that express markers of alternative activation (FIGS. 1A-1C). These polarized cells are capable of driving RGC and spinal cord axon regrowth in vitro as well as in vivo. Second, pro-regenerative Ly6G^{low} cells can also be induced in vivo by i.o. or i.p. administration of the fungal cell extract, zymosan, or its active ingredient β1,3-glucan. It has been previously shown that generation of pro-regenerative Ly6G^{low} cells in response to β1,3-glucan is dependent on signaling through its cognate receptor, dectin-1.

The next experiment magnifies the therapeutic potency of CD33⁺ HUCB cells/lines by polarizing them in vitro with immunomodulatory cytokines, dectin-1 agonists and/or prolyl hydroxylase inhibitors (that boost expression of HIF-1α), prior to clinical application, as unpublished work implicates hypoxia inducible factor-1α (HIF-1α) as a critical downstream mediator in the polarization of the RGC-protective/ON regenerative myeloid cells. Also, the donor cells are genetically engineered with lentiviral vectors that contain genes encoding growth factors, anti-apoptotic, and/or homing molecules. Of note, human CD34+ hematopoietic stem cells have been successfully transduced with lentiviruses carrying the wildtype version of genes that are mutated in X-lined adrenoleukodystrophy and β-thalassemia. Transplantation of those engineered cells into patients resulted in clinical improvements.

The study herein also modifies the CNS or ocular microenvironment to stabilize the phenotype of reparative HUCB myeloid subsets (via the forced expression of polarizing factors by retina resident cells) and promote their local retention (via forced expression of chemotactic factors). This can be accomplished via i.o., intathecal, intracerebral or intraventricular injection of recombinant adeno-associated virus (rAAV9) vectors carrying the genes of interest. The eye is a particularly suitable target for gene therapy because it is a contained compartment, permitting local delivery by direct injection, and it has a relatively immune privileged status. Gene therapy is well established in the treatment of patients with ocular diseases such as retinitis pigmentosa and Leber's congenital disease; over 30 rAAV gene therapies are registered on clinicaltrials.gov for trials in a range of ocular conditions. The experiment found that forced expression of recombinant murine IL-4 by resident retinal cells, following transduction with AAV9-IL-4 vectors, augments i.o. zymosan-induced ON axon regeneration in mice with ON crush injury (FIG. 1E).

Neuroprotective combination therapies can be more effective than individual agents in promoting neuron survival, as has been previously demonstrated in animal models of hypertensive glaucoma and ischemic optic neuropathy. In previous studies of the ON crush model, multimodal therapeutic approaches were more effective at inducing regeneration than single interventions, in some cases achieving RGC axon regrowth across the optic chiasm (such as i.o. zymosan injection combined with a CAMP analog and pten deletion). Herein indicates that the distinctive mechanisms of action of neuroregenerative HUCB myeloid cell infusion makes it a powerful component of a multimodal therapeutic regimen. In particular, HUCB infusions may synergize with agents that block cell-intrinsic or extrinsic suppressors of CNS axon growth, to amplify neuron survival and axon regrowth after injury.

### Neuroprotective/Pro-Regenerative Functions of HUCB Via In Vitro Polarization and Genetic Engineering.

In vitro polarization of HUCB to enhance therapeutic potency. Primary CD33⁺ HUCB cells or short term CD34⁺CD33⁺ HUCB cell lines are cultured (at 5×10⁵ cells/mL) for various lengths of time (between 6-24 hours) with recombinant human IL-4 (25 ng/mL), IL-10 (100 ng/mL), or TGF-β (100 ng/mL), and/or with the dectin-1 agonists β1,3-glucan (100 μg/mL) or scleroglucan (100 μg/mL), and/or with the prolyl-4-hydroxylase inhibitor, Dimethyloxalylglycine (DMOG, 250 uM). The polarized cells, as well as unpolarized counterparts, are harvested, washed and analyzed by the flow cytometric, transcriptomic and functional (neurite outgrowth and RGC viability) assays. Polarized cells that exhibit enhanced reparative and/or cytoprotective properties are adoptively transferred i.o. into RAG^{-/-} mice with ONC injury. Unpolarized cells are transferred into littermates for means of comparison. Viable RGC and regenerating ON axons are quantified in retinal whole mounts and ON sections, respectively, 4 weeks later. Mice are monitored with PERG at baseline and then weekly. Polarized cells that exhibit a superior therapeutic effect are injected i.o. at systematically reduced doses, and after increasingly delayed periods of time postinjury, to determine the lowest effective dose and broadest therapeutic window.

Genetic engineering of HUCB to enhance therapeutic potency. CD33⁺CD34^{+/-} HUCB cells/lines are genetically engineered to enhance their therapeutic potency, longevity and retention at sites of ocular injury. Recombinant lentiviral (LV) vectors are constructed to carry genes that encode neuroprotective or growth factors, anti-apoptotic molecules and/or chemotactic receptors, that CD33⁺CD34^{+/-} HUCB cells/lines either do not express or express at low levels. LV vectors are derived from the SINF-MU3-W-S vector backbone. Genes are driven by an EF1α promoter, inserted into SINF-MU3-W-S upstream of an encephalomyocarditis virus internal ribosome entry site (IRES)-green fluorescent protein (GFP) gene cassette. Other HUCB cells are transduced with GFP expression only vectors and used as controls. For transduction, isolated CD33⁺CD34^{+/-} HUCB cells/lines are cultured in StemSpan Myeloid Expansion media in 24 well nontissue culture-treated plates coated with RetroNectin recombinant fibronectin fragment (Takara Mirus Bio, Madison, WI) at 1×10⁶ cells/well. The cells are transduced for 24 hours with LV particles (2×10⁶ TU/mL; multiplicity of infection, 50) in the presence of protamine sulfate. Fresh medium is added and the cells are cultured for an additional 48-72 hours prior to harvest. Genetically engineered GFP+ HUCB cells are then FACS sorted and subjected to the same assays and adoptive transfer experiments outlined above for the in vitro polarized cells. Genetically engineered HUCB cells that have a superior therapeutic effect are injected i.o. at systematically reduced doses, and after increasingly delayed periods of time, post-ON crush injury to determine the lowest effective dose and broadest therapeutic window. Examples of genes that are inserted into the LV vectors include:

Nerve growth factor (NGF). NGF is a potent inducer of RGC axon regrowth in vivo. The current study found it to be expressed at very low or undetectable levels in pro-regenerative CD33⁺ HUCB cells and short-term lines. Thus, forced expression of NGF by HUCB cells can act synergistically with other growth factors naturally produced by those cells to augment their therapeutic potency.

CCR2. High levels of CCL2 are in the vitreal fluid of humanized mice with ON crush injury, as well as in i.o. zymosan injected mice with regenerating ON axons. Over-expression of CCR2 can increase the retention of donor HUCB cells at the site of ON injury.

BCL2L2. Lentiviral overexpression of BCL2L2 (which encodes the Bcl2 family member, Bcl-w) decreases the apoptosis of CD34⁺ HUCB derived megakaryocytes. Its effect on the life span of donor HUCB cells are assessed following adoptive transfer

The present work provides a polarizing cocktail that significantly enhances the neuroprotective and axon regenerative properties of primary CD33⁺ HUCB cells and CD33⁺CD34⁺ cell lines. Transduction of HUCB donor cells/lines with genes encoding certain growth factors, chemotactic receptors and/or anti-apoptotic molecules can enhance their pro-regenerative and cytoprotective effects, increase their efficiency, and extend their therapeutic window.

### Modulation of the CNS Microenvironment to Enhance HUCB Cell Therapy

Polarizing and chemotactic molecules are over-expressed in the posterior chamber of the eye via i.o. injection, or in the dorsal root ganglion via intra-sciatic nerve injection, of recombinant adeno-associated viral subtype 9 (rAAV9) vectors carrying genes that express those factors. The AAV9 subtype was chosen based on its strong tropism for RGC, horizontal cells and dorsal root ganglion neurons, and relatively high transduction efficiency in the retina and CNS. Loaded rAAV9-Transgene-GFP vectors or rAAV9-GFP only controls are injected intravitreally, just posterior to the limbus, or intraneurally, proximal to the dorsal root ganglion, into RAG1 KO mice 2-4 weeks prior to ONC or spinal cord traumatic injury and i.o. or intraneural injection of either CD33⁺ HUCB cells/lines, control cells or vehicle. (The actual timing of AAV injection is based on the results of experiments during which the kinetics of transgenic protein expression for each loaded rAAV9 vector can be determined). Outcome measures include counts of viable RGC in retinal whole mounts or neurons in dorsal root ganglia, densities of regenerating axons in ON or spinal cord sections, and PERG or motor evoked potential amplitudes, as described above. In addition, for ON crush experiments, vitreal fluid and cells are harvested from mice in all experimental groups at serial time points post-adoptive transfer, for analysis by proteomics (Luminex and ELISA), flow cytometry and transcriptomics.

Over-expression of recombinant murine IL-4 was observed in the posterior chamber of the eye, via transduction of retina resident cells with an AAV9-IL-4-GFP. Administration of this vector i.o. enhanced ON axon regeneration induced by local zymosan injection (FIG. 1E). High levels of IL-4 in the local environment stabilize the phenotype of pro-regenerative myeloid cells and convert uncommitted myeloid cells to that lineage. Human cytokines that enhance pro-regenerative HUCB polarization are overexpressed. The study also constructs rAAV vectors encoding cognate ligands of chemotactic receptors expressed on the surface of pro-regenerative HUCB cell subsets. If overexpression of certain polarizing or chemotactic factors can result in more robust neuron survival and/or axon regeneration, the dose of HUCB cells infused can be systematically lower, and administered following ON crush injury or spinal cord trauma after increasing time intervals, to determine the lowest effective dose and broadest therapeutic window. The study also determines if over-expression of selected chemotactic factors in the posterior chamber of the eye or dorsal root ganglion (DRG) drives the migration of labelled HUCB donor subsets to the vitreous or DRG, respectively, after they are administered intravenously. Depending on the results, the therapeutic efficacy of combining intravenous HUCB infusion with intraocular AAV9-chemokine gene therapy can be investigated in these models.

Overexpression of selected polarizing and chemotactic factors in the eye could boost the frequency of reparative HUCB cells maintained in the vitreous fluid over time, and enhance RGC survival and ON axon regeneration. Similarly, overexpression of selected polarizing and chemotactic factors in the DRG can boost the frequency of reparative HUCB cells maintained in the DRG over time, and enhance spinal cord neuronal survival and axon regeneration. Administration of gene therapy with AAV9 vectors encoding those factors can reduce the dose of HUCB infused, and extend the time post injury that HUCB infusions administered, while still attaining a robust therapeutic response.

### Example 9. Combination of H UCB Cellular Therapy with Interventions that Target Axon Growth Inhibitory Pathways Magnify the Therapeutic Response

The failure of CNS axon regeneration is due, in part, to the poor intrinsic growth capacity of mature neurons and the non-permissive CNS environment. The latter includes a panel of endogenous axon growth inhibitors (AGIs) that are contained in myelin. AGIs bind the Nogo receptor-1 (NgR1) on axons to induce nerve growth collapse and neurite outgrowth inhibition. Overexpression of an endogenous NgR1 antagonist, lateral olfactory tract usher substance (LOTUS), was recently shown to enhance RGC axon regeneration. A cell-intrinsic inhibitor of axon regeneration is phosphatase and tensin homolog (PTEN). AAV mediated shRNA silencing of PTEN also promotes ON axon regeneration. In this study, LOTUS is overexpressed, or PTEN expression is knocked down, in RGCs, secondary to intravitreal injection of an AAV-9 vector containing LOTUS or short hairpin RNA (shRNA) to silence PTEN, respectively. Control mice are injected i.o. with AAV9-GFP. The AAV9 vectors are administered 2 weeks prior to ON crush injury, based on previously published protocols. At the time of, or at serial time intervals following, crush injury, either pro-regenerative CD33⁺ HUCB cell subsets (at the lowest therapeutic dose, as determined above), control CD33⁻ HUCB or vehicle are injected by the intraocular route. Retina and optic nerves are harvested 4 weeks later for enumeration of viable RGC and regenerating ON axons, respectively. PERGs are performed weekly though week 4. LOTUS overexpression or PTEN knockdown can augment HUCB cell mediated RGC protection/ON axon regeneration. Then the lowest effective dose of HUCB and broadest therapeutic window are determined.

Multimodal therapy in mice with ON crush injury, that combines HUCB infusions with overexpression of LOTUS or knockdown of PTEN, can result in a higher RGC survival rate and increased frequency and average length of regenerating ON axons, compared with single interventions. Combination therapy can lower the minimum effective dose of HUCB cell infusions and broaden their therapeutic window.

rAAV vectors can activate TLR9. If rAAV9 vectors carries certain exogenous human genes are relatively immunogenic, short DNA oligonucleotides that antagonize TLR9 can be incorporated directly into the vector genome, and the vectors can be administered by subretinal injection. This strategy markedly reduced innate immune responses and enhanced gene expression in mouse and pig models following i.o. administration of modified rAAV vectors.

AAV based therapeutic approaches are not practical in many forms of optic neuropathy (including traumatic) due to the length of time it takes to reach maximal transgene expression post-transduction of retinal cells. These experiments, which involve prophylactic administration of rAAV vectors prior to ON injury, can demonstrate proof of principle of the therapeutic synergy between HUCB cell infusion and LOTUS overexpression or PTEN silencing. Treatment with biologics (ex. anti-Nogo blocking antibodies) or pharmacological agents (ex. PTEN antagonist peptides) that target the same axon inhibitory pathways, can be administered at the time of, or following, ON crush injury. Alternatively, new generation AAV vectors can be used, such as capsid residue tyrosine 444 to phenylalanine mutated single stranded AAVs, which enhance and accelerate transgene expression in the eye, and administered at the time of, or following, ON crush injury/HUCB cell infusion. Knockdown of the paired immunoglobulin-like receptor B (PirB) can also be applied in the retina with AAV PirB shRNA induces ON axon regeneration.

Statistical Analysis. Estimates are based on data to infer coefficients of variation (CV) or standardized effect sizes based on prior experience and designed to achieve ≥80% power. In prior publications with similar data, feasibility uses appropriate transformations (e.g., logarithm) to produce normal-like data for testing in proposed studies. Outcome measures (e.g., RGC frequency, RGC axon density, and PERG amplitudes) are analyzed by one-way ANOVA followed by Tukey's post hoc test using Graphpad 8.0 (Prism). Assays from flow cytometry, ELISA, RGC cultures and qPCR data are analyzed using a two-sample t-test for two group comparison or ANOVA with post hoc test for multiple-group comparisons. Differential gene expression analyses of scRNAseq data, to identify cell type specific genes, are performed using the non-parametric Wilcoxon rank sum test with Bonferroni adjusted p value less than 0.05. In order to compare the average gene expression of genes in the cell types identified in the single cell datasets, heat maps using Pearson's correlation values are generated. Volcano plots of differentially expressed genes are generated using R package EnhancedVolcano. Gene set enrichment analysis is performed using the Enrichr tool.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. An engineered cell, wherein the engineered cell has a phenotype of CD33+.

2. The engineered cell of claim 1, wherein the engineered cell has a phenotype of CD33+CD34+ or CD33+CD34dim.

3. The engineered cell of claim 1, wherein the engineered cell is an umbilical cord blood (HUBC) cell.

4. The engineered cell of claim 1, wherein the engineered cell is derived from a bone marrow cell.

5. The engineered cell of claim 4, wherein the bone marrow cell is contacted with IL-4 and/or granulocyte colony-stimulating factor (G-CSF) to create the engineered cell.

6. The engineered cell of claim 5, wherein the bone marrow cell is contacted with IL-4 and/or G-CSF in vitro or ex vivo to create the engineered cell.

7. The engineered cell of claim 1, wherein the engineered cell is derived from a stem cell obtained from a human, wherein the human was previously administered with G-CSF.

8. The engineered cell of claim 7, wherein the stem cell is contacted with IL-4 and/or G-CSF to create the engineered cell.

9. The engineered cell of claim 8, wherein the stem cell is contacted with IL-4 and/or G-CSF in vitro or ex vivo to create the engineered cell.

10. The engineered cell of claim 1, wherein the engineered cell is enhanced in the expression of a neuroprotective molecule or growth factor as compared to a reference control.

11. The engineered cell of claim 10, wherein the neuroprotective molecule is selected from the group consisting of nerve growth factor β (NGF β), brain derived growth factor (BDGF), ciliary neurotrophic factor receptor (CNTF), glial derived neurotrophic factor (GDNF), transforming growth factor-β (TGF β), transforming growth factor-α (TGF α), interleukin-4 (IL-4), heparin-binding epidermal growth factor (HB-EGF), fibroblast growth factors (FGFs), granulocyte-colony stimulating factor (G-CSF) and insulin-like growth factor 1 (IGF-1) and insulin-like growth factor 2 (IGF-2), and insulin-like growth factor 1 (IGF-1).

12. The engineered cell of claim 1, wherein the engineered cell is enhanced in the expression of a chemotactic receptor as compared to a reference control.

13. The engineered cell of claim 12, wherein the chemotactic receptor comprises CCR2, CCR3, CXCR2 or CXCR3.

14. The engineered cell of claim 1, wherein the engineered cell is enhanced in the expression of an anti-apoptotic molecule as compared to a reference control.

15. The engineered cell of claim 14, wherein the anti-apoptotic molecule comprises BCL2L2.

16. A composition comprising the engineered cell of claim 1.

17-23. (canceled)

24. A method of treating a neurological disorder in a subject, comprising administering to the subject a therapeutically effective amount of the engineered cell of claim 1.

25-35. (canceled)

36. A method of improving the regeneration of a nerve cell in a subject, comprising administering to the subject a therapeutically effective amount of the engineered cell of claim 1.

37-47. (canceled)

48. An extracellular vesicle derived from the engineered cell of claim 1.

49. A method of treating a neurological disorder in a subject, comprising administering to the subject a therapeutically effective amount of the extracellular vesicle of claim 48.

50. (canceled)


SEQUENCE LISTING (3 sequences)

SEQ ID NO: 1 - DNA, 19 bp, synthetic construct
  source 1..19: mol_type = other DNA
tgaacattggaatagtttc

SEQ ID NO: 2 - DNA, 19 bp, synthetic construct
  source 1..19: mol_type = other DNA
ttgaactgctagcctctgg

SEQ ID NO: 3 - DNA, 19 bp, synthetic construct
  source 1..19: mol_type = other DNA
acaacaagcagtgacagcg